(19) United States
(12) Patent Application Publication
(10) Pub. No.: US 2021/0128509 A1
(43) Pub. Date: May 6, 2021

(54) USE OF MAGNETIC RESONANCE SPECTROSCOPY TO CALIBRATE AND SELECT DOSES, FORMULATIONS, AND DEVICES FOR INTRA-NASAL ADMINISTRATION OF N-ACETYLCYSTEINE

(71) Applicant: Neuronasal, Inc., Wexford, TX (US)

(72) Inventor: Douglas A. GREENE, Basking Ridge, NJ (US)

(21) Appl. No.: 17/089,192

(22) Filed: Nov. 4, 2020

Related U.S. Application Data
(60) Provisional application No. 62/930,473, filed on Nov. 4, 2019.

(51) Int. Cl. A61K 31/198; A61K 9/00; G01N 24/08; G01N 33/15

(52) U.S. Cl. CPC A61K 31/198; A61K 9/0043; G01N 33/15; G01N 24/08; A61K 9/008

(57) ABSTRACT

The present disclosure describes methods of administering N-acetylcysteine (NAC) via intranasal administration. The effect of intranasal NAC administration can be monitored using an analytical technique, for example, magnetic resonance spectroscopy (MRS). In some embodiments, intranasal NAC can be used to treat a condition. In some embodiments, MRS can be used to monitor the effect of intranasal NAC administration or to modify the dosage of intranasal NAC administration to treat a condition.

### CROSS-REFERENCE

This application claims the benefit of U.S. Provisional Application No. 62/930,473 filed Nov. 4, 2019, which is incorporated herein by reference in its entirety.

### BACKGROUND

NAC is a precursor of L-cysteine that results in glutathione elevation biosynthesis. NAC is a powerful antioxidant that acts directly as a scavenger of free radicals, for example, oxygen free radicals. NAC can be used as a treatment option for disorders resulting from the generation of free oxygen radicals. NAC has a range of pleotropic salutary effects on acute and chronic central nervous system (CNS) disorders. Methods of administering NAC and quantifying the effects of NAC on the brain are necessary to improve the therapeutic use of NAC.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

Disclosed herein is a method of treating a condition comprising: a) administering to a subject in need thereof a therapeutically-effective amount of a therapeutic agent, wherein the administering is intranasal; and b) after the administering, quantifying a concentration of glutathione in a brain region by magnetic resonance spectroscopy.

### BRIEF DESCRIPTION OF THE DRAWINGS

FIG. 1 illustrates the biological activities of N-acetylcysteine.

FIG. 2 illustrates a schematic of the single ascending dose study for intranasal administration of N-acetylcysteine.

FIG. 3 illustrates a schematic for a device comparison study.

FIG. 4 illustrates a schematic to study the effect of the subject's position during IP administration.

FIG. 5 illustrates a schematic to study the effect of repeat dosing of intranasal N-acetylcysteine administration.

FIG. 6 illustrates a schematic to compare the effects of intranasal, intravenous, and oral administration of N-acetylcysteine.

FIG. 7A shows the change in GSH/water (I.U.) ratio in the dorsolateral prefrontal cortex (DLPF), occipital lobe (OCC), and striatum regions of the brain. FIG. 7B shows the percent change in GSH/water ratio in the DLPF, OCC, and striatum.

FIG. 8A shows the change in GSH/creatine ratio in the dorsolateral prefrontal cortex (DLPF), occipital lobe (OCC), and striatum regions of the brain. FIG. 8B shows the percent change in GSH/creatine ratio in the DLPF, OCC, and stratum regions of the brain.

FIG. 9A shows the N-acetyl aspartate (NAA)/water ratio in the dorsolateral prefrontal cortex (DLPF), occipital lobe (OCC), and striatum regions of the brain. FIG. 9B shows the percent change in the NAA/water ratio in the dorsolateral prefrontal cortex (DLPF), occipital lobe (OCC), and striatum regions of the brain.

FIG. 10A shows the N-acetyl aspartate (NAA)/creatine ratio in the dorsolateral prefrontal cortex (DLPF), occipital lobe (OCC), and striatum regions of the brain. FIG. 10B shows the percent change in the NAA/creatine ratio in the dorsolateral prefrontal cortex (DLPF), occipital lobe (OCC), and striatum regions of the brain.

FIG. 11A shows the percent change of GSH/creatine and percent change of NAA/creatine in the dorsolateral prefrontal cortex (DLPF) region of the brain. FIG. 11B shows the percent change of GSH/creatine and percent change of NAA/creatine in the occipital lobe (OCC) region of the brain. FIG. 11C shows the percent change of GSH/creatine and percent change of NAA/creatine in the striatum region of the brain.

### DETAILED DESCRIPTION

Concussions, also known as mild traumatic brain injuries (mTBIs), are transient and clinically detectable alterations in brain function resulting from mechanical insult transmitted to the brain. The global incidence of mTBI is approximately 42 million persons per year, with 100 to 300 per 100,000 individuals seeking medical attention annually. The risk of mTBI, as well as repetitive mTBI and sub-concussive injuries, is increased for subpopulations such as military personnel, athletes and victims of domestic abuse. Civilian mTBI may result from blunt trauma sustained in accidents, assaults, or participation in athletic activities. The Centers for Disease Control (CDC) estimates that there 1.6 to 3.8 million sports- and recreation-related concussions each year in the US. The direct and indirect costs attributable to concussions have been estimated at over $17 billion annually in the U.S. alone.

For military personnel, blast injury is a frequent cause of concussion and more severe head injuries. Seventy-five percent of the head injuries due to explosive blasts are classified as mild. The incidence of military mTBI between 1997 and 2007 was approximately 6.6 per 1000 person-years of service, and 17% of Army veterans returning from Iraq or Afghanistan reported having sustained concussions, with more than half reporting two or more sustained concussions.

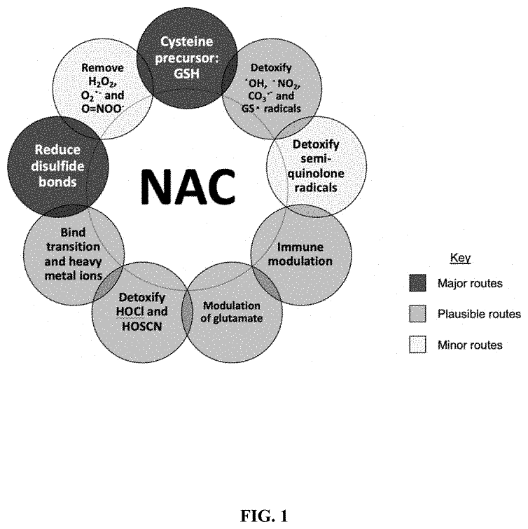

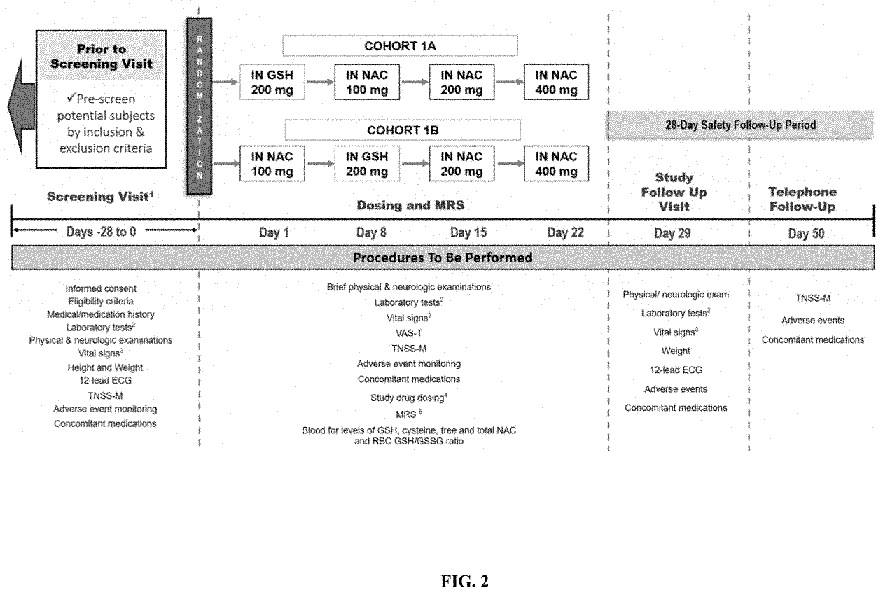

N-acetylcysteine (NAC) is synthetic small-molecule. FIG. 1 illustrates the biological activities of N-acetylcysteine. NAC has a range of pleotropic salutary effects on acute and chronic central nervous system (CNS) disorders through a variety of biochemical and pharmacological mechanisms of action, including quenching of reactive oxygen species (ROS), chelation of oxidative reactive metal ions, anti-inflammation, and neuromodulation via the cystine-glutamate antiporter. NAC can also increase the concentration and bioavailability of the endogenous antioxidant glutathione (GSH), anti-excitotoxic activity, and heavy metal-chelating activity.

Disclosed herein are methods of intranasally administering a compound of the disclosure and quantifying neurometabolites within the CNS using an analytical technique. Disclosed herein is a method of treating a condition comprising: a) administering to a subject in need thereof a therapeutically-effective amount of a therapeutic agent, wherein the administering is intranasal; and b) after the administering, quantifying a concentration of NAC or a NAC-neurometabolite in a brain region by magnetic resonance spectroscopy. Disclosed herein is a method of treating a condition comprising: a) administering to a subject in need thereof a therapeutically-effective amount of a therapeutic agent, wherein the administering is intranasal; and b) after the administering, quantifying a concentration of glutathione in a brain region by magnetic resonance spectroscopy. In some embodiments, the therapeutic agent is NAC. In some embodiments, the therapeutic agent is NACA. In some embodiments, the therapeutic agent is a NAC derivative or a pharmaceutically-acceptable salt thereof. In some embodiments, the NAC derivative is GSH. In some embodiments, the therapeutic agent is a NAC congener or a pharmaceutically-acceptable salt thereof. In some embodiments, the therapeutic agent is a NAC dendrimer (D-NAC) or a pharmaceutically-acceptable salt thereof.

In some embodiments, the disclosure provides methods of quantifying NAC-derived neurometabolites within the CNS using magnetic resonance spectroscopy (MRS). In some embodiments, MRS is used to determine the pharmacokinetics and pharmacodynamics of NAC, NACA, a NAC metabolite, NAC congener, or D-NAC, or a pharmaceutically-acceptable salt thereof, in healthy volunteers by quantifying NAC-derived neurometabolites. In some embodiments, MRS is used to determine safe and tolerable doses of intranasal NAC, NACA, a NAC metabolite, NAC congener, or D-NAC, or a pharmaceutically-acceptable salt thereof, by quantifying NAC-derived neurometabolites.

The disclosure also describes methods of treating various brain disorders involving oxidative stress or reactive oxygen species (ROS) that cause inflammation, excitotoxicity, and cell death. In some embodiments, tolerable dose-volumes of aqueous solutions of NAC, NACA, a NAC metabolite, NAC congener, or D-NAC, or a pharmaceutically-acceptable salt thereof, can be administered to achieve sufficient measures of brain bioactivity. In some embodiments, tolerable dose-volumes of aqueous solutions of NAC, NACA, a NAC metabolite, NAC congener, or D-NAC, or a pharmaceutically-acceptable salt thereof, cannot achieve sufficient measures of bioactivity, and can require the use of alternative delivery techniques and/or formulations. In some embodiments, novel formulation-device combinations are tested by monitoring MRS-GSH levels in the brain.

The methods described herein can administer NAC, NACA, a NAC metabolite, NAC congener, or D-NAC, or a pharmaceutically-acceptable salt thereof. In some embodiments, NAC, NAC amide, a NAC derivative, a NAC metabolite, or a NAC congener thereof are administered intranasally. In some embodiments, NAC, NAC amide, a NAC derivative, a NAC metabolite, or a NAC congener thereof are administered intranasally using an atomizer, for example, a Teleflex LMA® MAD Nasal™ Intranasal mucosal atomization device. In some embodiments, NAC, NACA, a NAC metabolite, NAC congener, or D-NAC, or a pharmaceutically-acceptable salt thereof, is administered intranasally using a nasal pump, for example, Aptar CPS 5-mL Nasal Pump.

In some embodiments, the methods of the disclosure can treat a brain condition. In some embodiments, the brain condition is mild traumatic brain injury (mTBI). In some embodiments, the brain condition is cancer. In some embodiments, the brain condition is a central nervous system disorder. In some embodiments, the CNS disorder is Parkinson's disease.

### Mechanism of Action

NAC, NACA, a NAC metabolite, NAC congener, or D-NAC, or a pharmaceutically-acceptable salt thereof, can treat a condition by acting as a cysteine or GSH precursor. GSH is an endogenous compound that is essential to intracellular defenses against oxidative damage. GSH is a free radical scavenger and a key component of maintaining the redox state of cells in the CNS. GSH contains three amino acids: glutamate, glycine and cysteine. Cysteine is present at the lowest concentration intracellularly. With oxidative stress, cysteine concentration is rate-limiting for the synthesis of GSH, which therefore becomes depleted because of concussion-induced excitotoxicity and the resultant changes in cell metabolism.

The major mechanism of action for NAC is the ability of NAC-derived cysteine to serve as a precursor for the synthesis and replenishment of cellular GSH stores. The strength of the effect of NAC on GSH concentration is controlled in part by the degree of endogenous cellular cysteine availability and the degree of endogenous GSH depletion. Correction of cellular GSH depletion is a major component of NAC's putative neuroprotective effects in psychiatric and neurodegenerative disorders. The neuroprotective effects of NAC depend on the extent to which NAC and/or NAC-derived reduced sulfhydryl equivalents can access the central nervous system to augment endogenous antioxidant activity.

NAC can also reduce disulfide bonds in proteins and disrupt ligand bonding and alter protein structures. NAC's ability to reduce disulfide bonds in mucolytic proteins accounts for the action of NAC as an effective mucolytic agent. NAC can also act as a glutamatergic modulator. Cysteine in the nervous system can assist in the regulation of neuronal intracellular and extracellular exchange of glutamate through the cystine-glutamate antiporter preferentially located on glial cells. In response to NAC-derived cystine, glial cells release glutamate into the extracellular space stimulating inhibitory metabotropic glutamate receptors on glutamatergic nerve terminals and thereby reducing the synaptic release of glutamate thereby affecting glutamatergic synaptic function and potentially ameliorating post-injury neuro-excitotoxicity.

NAC can act as a free radical scavenger and directly quench free radicals such as hydroxyl, nitrogen dioxide, carbonate and thiyl radicals and detoxify semiquinones, hypochlorous acid, and nitrosyl hydride. Under physiological conditions NAC does not react with nitric oxide, superoxide, hydrogen peroxide or peroxynitrite. NAC can act as an anti-inflammatory agent. NAC has demonstrated immunomodulatory activity in a variety of experimental and clinical pro-inflammatory conditions, including human autoimmune disorders such as Sjogren's syndrome and systemic lupus erythematosus.

### Compounds of the Invention

N-acetylcysteine (NAC) is a glutathione prodrug that is used to treat acetaminophen-induced liver failure and to loosen thick mucus individuals with cystic fibrosis or chronic obstructive pulmonary disease. NAC can be taken intravenously, by mouth, or inhaled as a mist. Common side effects of NAC include nausea and vomiting when NAC is administered orally. NAC can also cause skin redness and itching and a non-immune type of anaphylaxis. NAC has multiple putative targets of action, and NAC has poor penetration into the CNS. NAC has been reported to cause nausea and vomiting, induce bronchospasm, slow blood clotting, and induce neurotoxicity in a dose-dependent manner. These issues can be problematic for patients with hemorrhagic stroke.

The present disclosure describes the use of at least one compound or a pharmaceutically-acceptable salt thereof to treat a condition. In some embodiments, the compound is N-acetylcysteine (NAC), NAC amide (NACA), NAC derivative, NAC metabolite, NAC congener, or NAC dendrimer (D-NAC), or a pharmaceutically-acceptable salt thereof. In some embodiments, the compound is a NAC prodrug or a pharmaceutically-acceptable slat thereof. In some embodiments, the compounds is NAC. In some embodiments, the compound is a NAC derivative. In some embodiments, the NAC derivative is GSH.

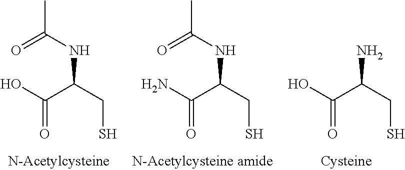

In some embodiments, the compound is a NAC dendrimer. Dendrimer-NAC (D-NAC) is a dendrimer conjugate where NAC is covalently bound to the surface of a dendrimer by disulfide linkages. In some embodiments, D-NAC comprises a polyamidoamine (PAMAM) hydroxyl dendrimer. In some embodiments, D-NAC comprises a polyglycerol sulfate dendrimer. In some embodiments, D-NAC comprises a polyamine dendrimer. In some embodiments, D-NAC comprises a polyamide dendrimer. In some embodiments, D-NAC comprises a linker. In some embodiments, GABA comprises a gamma-aminobutyric acid (GABA) linker. In some embodiments, D-NAC comprises a succinimidyl 3-(2-pyridyldithio)propionate (SPDP) linker.

In some embodiments, D-NAC has the formula:

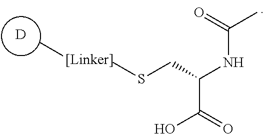

In some embodiments, D-NAC has the formula:

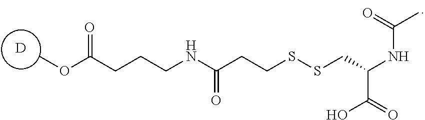

### Purity of Compounds of the Invention

Any compound of the disclosure can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Method of Detection and Clinical Assessment Tools

Magnetic resonance spectroscopy (MRS) is a technique associated with magnetic resonance imaging (MRI). MRS, also known as nuclear magnetic resonance (NMR) spectroscopy, is a non-invasive, ionizing-radiation-free analytical technique that can detect and measure metabolic changes in an organ, for example, the brain. MRS acquires signals from hydrogen protons in water and fat, which are approximately a thousand times more abundant than the molecules detected with MRS. In some embodiments, MRS is used to acquire a signal from a single localized region of the brain, referred to as a “voxel”. In some embodiments, MRS can be used to determine a relative concentration of a biochemical in the region of the brain. In some embodiments, MRS can be used to determine a physical property of a region of the brain.

In some embodiments, MRS can be used to determine a relative concentration of a metabolite in the region of the brain. In some embodiments, the methods of the disclosure measure the concentration of a neurometabolic marker after intranasal administration of NAC, NACA, NAC derivative, NAC metabolite, NAC congener, or D-NAC, or a pharmaceutically-acceptable salt thereof. In some embodiments, the methods of the disclosure measure a concentration of GSH after intranasal administration of NAC. In some embodiments, the methods of the disclosure measure a concentration change of GSH after intranasal administration of NAC.

Also disclosed herein are methods of treating a brain disorder by monitoring absorption of a compound of the disclosure. In some embodiments, NAC, NACA, NAC derivative, NAC metabolite, NAC congener, or D-NAC, or a pharmaceutically-acceptable salt thereof is administered to a subject, and dosing of NAC, NACA, NAC derivative, NAC metabolite, NAC congener, or D-NAC, or a pharmaceutically-acceptable salt thereof is changed based on MRS analysis of the brain to determine the concentration of a neurometabolite after intranasal administration of NAC, NACA, NAC derivative, NAC metabolite, NAC congener, or D-NAC, or a pharmaceutically-acceptable salt thereof. In some embodiments, the NAC derivative is GSH.

In some embodiments, the neurometabolic marker is a NAC neurometabolite. In some embodiments, the neurometabolite is N-acetyl aspartate, lactate, glutamate, gamma-aminobutyric acid, or glutathione. In some embodiments, the NAC neurometabolite is glutathione. In some embodiments, the NAC neurometabolite is N-acetyl aspartate.

In some embodiments, the methods of the disclosure can detect the action of NAC, NACA, NAC derivative, NAC metabolite, NAC congener, or D-NAC, or a pharmaceutically-acceptable salt thereof as a cysteine precursor. In some embodiments, the NAC, NACA, NAC derivative, NAC metabolite, NAC congener, or D-NAC, or a pharmaceutically-acceptable salt thereof can increase GSH synthesis. In some embodiments, the NAC, NACA, NAC derivative, NAC metabolite, NAC congener, or D-NAC, or a pharmaceutically-acceptable salt thereof can modulate gamma-aminobutyric acid (GABA) neurotransmission. In some embodiments, the action of NAC, NACA, NAC derivative, NAC metabolite, NAC congener, or D-NAC, or a pharmaceutically-acceptable salt thereof as cysteine precursors can be assessed by measuring a change in GSH by quantifying a β-CH2 MRS signature from cysteine moieties.

The methods of the disclosure can further comprise Meshcher-Garwood Point Resolved Spectroscopy (MEGA-PRESS). In some embodiments, the methods of the disclosure can use MEGA-PRESS to separately but simultaneously measure post-drug administration changes in the β-CH2 MRS signature common to both NAC and GSH. In some embodiments, the methods of the disclosure can use MEGA-PRESS to determine the relative post-dose increase in β-CH2 MRS signature. In some embodiments, the methods of the disclosure can use MEGA-PRESS to determine the conversion of NAC, NACA, NAC derivative, NAC metabolite, NAC congener, or D-NAC, or a pharmaceutically-acceptable salt thereof to NAC-metabolite in a region-specific fashion. In some embodiments, the methods of the disclosure can use MEGA-PRESS to determine the conversion of NAC, NACA, NAC derivative, NAC metabolite, NAC congener, or D-NAC, or a pharmaceutically-acceptable salt thereof to GSH in a region-specific fashion. In some embodiments, the methods of the disclosure can use MEGA-PRESS to determine the conversion of NAC, NACA, NAC derivative, NAC metabolite, NAC congener, or D-NAC, or a pharmaceutically-acceptable salt thereof to GSH in a time-specific fashion.

In some embodiments, the methods of the disclosure detect and quantify a concentration change in a NAC-neurometabolite after administration of NAC, NACA, NAC derivative, NAC metabolite, NAC congener, or D-NAC, or a pharmaceutically-acceptable salt thereof to optimize at least on delivery parameter of administering NAC, NACA, NAC derivative, NAC metabolite, NAC congener, or D-NAC, or a pharmaceutically-acceptable salt thereof. In some embodiments, the delivery parameter is dose. In some embodiments, the delivery parameter is dose interval. In some embodiments, the delivery parameter is a dose delivery system.

In some embodiments, the methods of the disclosure detect and quantify a concentration change in a NAC-neurometabolite after administration of NAC, NACA, NAC derivative, NAC metabolite, NAC congener, or D-NAC, or a pharmaceutically-acceptable salt thereof to optimize the presence of NAC in the brain. In some embodiments, the methods of the disclosure detect and quantify a concentration change in a NAC-neurometabolite after administration of NAC, NACA, NAC derivative, NAC metabolite, NAC congener, or D-NAC, or a pharmaceutically-acceptable salt thereof to optimize the presence of GSH in the brain. In some embodiments, the methods of the disclosure detect and quantify a concentration change in a NAC-neurometabolite after administration of NAC, NACA, NAC derivative, NAC metabolite, NAC congener, or D-NAC, or a pharmaceutically-acceptable salt thereof to optimize the presence of NAC and GSH in the brain.

In some embodiments, the methods of the disclosure detect and quantify a concentration change in a NAC-neurometabolite after administration of NAC, NACA, NAC derivative, NAC metabolite, NAC congener, or D-NAC, or a pharmaceutically-acceptable salt thereof to optimize the presence of NAC in a region of the brain. In some embodiments, the methods of the disclosure detect and quantify a concentration change in a NAC-neurometabolite after administration of NAC, NACA, NAC derivative, NAC metabolite, NAC congener, or D-NAC, or a pharmaceutically-acceptable salt thereof to optimize the presence of GSH in a region of the brain. In some embodiments, the methods of the disclosure detect and quantify a concentration change in a NAC-neurometabolite after administration of NAC, NACA, NAC derivative, NAC metabolite, NAC congener, or D-NAC, or a pharmaceutically-acceptable salt thereof to optimize the presence of NAC and GSH in a region of the brain.

The methods of the disclosure can further comprise obtaining biological samples for analysis. In some embodiments, the method further comprises quantifying an amount of free NAC in a plasma sample. In some embodiments, the method further comprises quantifying an amount of total NAC in a plasma sample. In some embodiments, the method further comprises quantifying an amount of plasma GSH. In some embodiments, the method further comprises quantifying a ratio of reduced GSH to oxidized GSH (GSH/GSSG). In some embodiments, the method further comprises quantifying an amount of NAC or a NAC metabolite in a cerebrospinal fluid sample.

Several tools can be utilized to diagnose and assess the clinical and neuropsychological features of a brain condition, for example, mild traumatic brain injury. In some embodiments, standard physical and neurological examinations, and neuropsychometric batteries and scales with broader applicability (e.g., Glasgow coma scale) can be used to diagnose and assess a subject with a CNS condition.

Post-concussion symptom score (PCSS): The PCSS score consists of 22 items that evaluate symptoms on a 7-point scale. 0 correlates to no symptoms, and 6 correlates to severe symptoms. PCSS scores have utility for subjects ages 11 and above in identifying individuals with clinically-diagnosed concussion, and in predicting prolonged recovery. PCSS scores have also demonstrated test-retest reliability.

Graded symptom checklist (GSC): The GSC consists of 16 items scored on a 7-point scale. The GSC scale is applicable to subjects ages 13 and above, and incorporates a three-factor structure (cognitive, somatic, and neurobehavioral). The GSC scale has demonstrated internal validity, test-retest reliability, and convergent validity with respect to balance and cognitive performance.

Standardized concussion assessment tool (SCAT): SCAT is a standardized tool that is used by healthcare professionals, and incorporates other assessment scales, such as GCS, Maddocks questions for memory assessment, PCSS, and other neurological and cognitive tests.

Immediate post-concussion assessment and cognitive testing (ImPACT): ImPACT is a computerized test battery with 3 components, such as demographic data, neuropsychological testing, and PCSS. ImPACT has the advantage of including assessments of cognition (e.g., attention, processing speed, impulsivity, and reaction time). In a combination with a scale for mTBI symptoms, ImPACT has a sensitivity of 81.9%, and a specificity of 89.4%. ImPACT is not subject to substantial practice effects.

King-Devick Scale: The King-Devick scale is a brief test administered acutely following head injury in which the subject must read patterns of letters and numbers on test cards. The King-Devick scale assess language, attention, and eye movements, all of which can be impaired in a CNS condition, for example, concussion. The test-retest reliability of the King-Devick scale over a period of 1-2 years compares is comparable to other standard assessment methods.

Biomarkers and imaging: Electrophysiological techniques, imaging techniques, and blood tests can be used to assess the CNS condition of a subject. Event-related potentials (EPRs) can be used to evaluate computer-processed electroencephalogram (EEG) signals time-locked to a perpetual or cognitive task. In some embodiments, computed tomography (CT) and magnetic resonance imaging (MRI) can be used to diagnose or track the progress of a CNS condition. In some embodiments, diffusion tensor imaging can be used to diagnose or track the progression of a CNS condition.

### Methods of Administration

Compounds of the disclosure can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration. In some embodiments, a therapeutically-effective amount of a compound of the disclosure can be administered intranasally.

A compound or pharmaceutical composition of the disclosure can be administered in a local manner, for example, intranasally. Intranasal administration is a route of administration where drugs are insufflated through the nose. In some embodiments, intranasal administration can administer a compound or pharmaceutical composition of the disclosure topically. In some embodiments, intranasal administration can administer a compound or pharmaceutical composition of the disclosure systemically.

The nasal cavity's easily accessible, rich vascular plexus permits topically administered drugs to rapidly achieve therapeutically effective blood levels while avoiding intravenous catheters. In some embodiments, nasal administration can be used to deliver a compound of pharmaceutical composition of the disclosure to the blood stream. In some embodiments, nasal administration can be used to deliver a compound or pharmaceutical composition of the disclosure to the blood. In some embodiments, nasal administration delivers a compound or pharmaceutical composition of the disclosure to the blood, which then enters the brain. Intranasal administration of a compound or pharmaceutical composition disclosed herein avoids gastrointestinal destruction and hepatic first pass metabolism, which allows the compound or pharmaceutical composition to be most cost-effectively and rapidly bioavailable compared to oral administration. In some embodiments, intranasal administration of a compound or pharmaceutical composition of the disclosure can make the bioavailability of the compound or pharmaceutical composition more predictable compared to oral administration.

In some embodiments, intranasal administration of a compound or pharmaceutical composition of the disclosure can have a rate of absorption that is greater than subcutaneous or intramuscular administration. In some embodiments, intranasal administration of a compound or pharmaceutical composition of the disclosure can have a resulting plasma concentration that is greater than subcutaneous or intramuscular administration. In some embodiments, intranasal administration of a compound or pharmaceutical composition of the disclosure can rapidly achieve therapeutic brain and spinal cord drug concentrations.

A liquid pharmaceutical composition of the disclosure can be administered to a subject intranasally using a device. In some embodiments, a liquid formulation can be delivered as drops with a pipette. In some embodiments, a liquid formulation can be delivered with a catheter and a squirt tube, for example, a rhinyl catheter and a squirt tube. In some embodiments, a liquid formulation can be delivered using a squeeze bottle.

In some embodiments, a liquid formulation can be administered intranasally using a mechanical spray pump. In some embodiments, a liquid formulation can be intranasally administered using a metered-dose spray pump. In some embodiments, a liquid formulation can be delivered using a single-dose or duo-dose spray device. In some embodiments, a liquid formulation can be delivered using a nasal pressurized metered-dose inhaler (pMDI).

In some embodiments, a liquid formulation can be administered intranasally using a gas-driven spray system or atomizer. In some embodiments, a liquid formulation can be administered intranasally using a nitrogen gas-driven system. In some embodiments, a liquid formulation can be administered intranasally using a powdered nebulizer or atomizer. In some embodiments, a liquid formulation can be administered intranasally using a VibrENT pulsation membrane nebulizer. In some embodiments, a liquid formulation can be administered intranasally using an Aeroneb Solo vibrating mesh nebulizer. In some embodiments, a liquid formulation can be administered intranasally using a ViaNase atomizer. In some embodiments, a liquid formulation can be administered intranasally using a Teleflex LMA® MAD Nasal™ intranasal mucosal atomization device. In some embodiments, a liquid formulation can be administered intranasally using a Aptar CPS 5-mL nasal pump.

In some embodiments, a powder formulation can be administered intranasally using a device. In some embodiments, a powder formulation can be administered intranasally using a nasal powder inhaler. In some embodiments, a powder formulation can be administered intranasally using a nasal powder sprayer. In some embodiments, a powder formulation can be administered intranasally using a nasal powder insufflator. In some embodiments, a powder formulation can be administered intranasally using a breath-powered Bi-Directional technology device.

The compounds or pharmaceutical compositions of the disclosure can be administered in various positions. In some embodiments, the compounds or pharmaceutical compositions of the disclosure can be administered to the subject in the supine position. In some embodiments, the compounds or pharmaceutical compositions of the disclosure can be administered to the subject in the seated position.

### Pharmaceutical Compositions

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. A pharmaceutical composition of the invention can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives. In some embodiments, pharmaceutical compositions of the disclosure can comprise a stabilizer. In some embodiments, the stabilizer is Captisol®, Monosteol™, Vivapur® MCG 591P, Vivapur® MCG 611P, Vivapur® MCG 811P, Neosorb sorbitol solution sweetener coating, HiCel MCG 581, HiCel MCG591, or HiCel MCG611.

In some embodiments, the pharmaceutical compositions of the disclosure can comprise an absorption enhancer. In some embodiments, the absorption enhancer is a peptide or a protein. In some embodiments, the absorption enhancer is calcitonin, desmopressin, insulin, leuprolide, or octreotide. In some embodiments, the absorption enhancer is a non-peptide macromolecule. In some embodiments, the absorption enhancer is heparin, low-molecular weight heparin, enoxaparin, fondaparinux, an oligonucleotide, or vancomycin. In some embodiments, the absorption enhancer is a hydrophilic small molecule. In some embodiments, the absorption enhancer is an aminoglycoside, amikacin, gentamycin, amphotericin B, or bisphosphonate.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A pharmaceutical composition of the disclosure can be in the form of an aqueous solution. In some embodiments, the pharmaceutical composition can comprise from about 5% to about 10%, from about 10% to about 15%, from about 15% to about 20%, from about 20% to about 25%, or from about 25% to about 30% of NAC, NACA, NAC derivative, NAC metabolite, NAC congener, or D-NAC, or a pharmaceutically-acceptable salt thereof in an aqueous solution. In some embodiments, a pharmaceutical composition of the disclosure can comprise about 5%, about 10%, about 15%, about 20%, about 25%, or about 30% of NAC, NACA, NAC derivative, NAC metabolite, NAC congener, or D-NAC, or a pharmaceutically-acceptable salt thereof in an aqueous solution. In some embodiments, a pharmaceutical composition of the disclosure can comprise about 10%, about 11%, about 12%, about 13%, about 14%, about 15%, about 16%, about 17%, about 18%, about 19%, or about 20% of NAC, NACA, NAC derivative, NAC metabolite, NAC congener, or D-NAC, or a pharmaceutically-acceptable salt thereof in an aqueous solution. In some embodiments, a pharmaceutical composition of the disclosure can comprise about 15% of NAC, NAC, NACA, NAC derivative, NAC metabolite, NAC congener, or D-NAC, or a pharmaceutically-acceptable salt thereof in an aqueous solution. In some embodiments, a pharmaceutical composition of the disclosure can comprise about 20% of NAC, NACA, NAC derivative, NAC metabolite, NAC congener, or D-NAC, or a pharmaceutically-acceptable salt thereof in an aqueous solution. In some embodiments, a pharmaceutical composition of the disclosure can comprise about 25% of NAC, NACA, NAC derivative, NAC metabolite, NAC congener, or D-NAC, or a pharmaceutically-acceptable salt thereof in an aqueous solution.

A pharmaceutical composition of the disclosure can be in the form of a dry powder. In some embodiments, the pharmaceutical composition can comprise from about 5% to about 10%, from about 10% to about 15%, from about 15% to about 20%, from about 20% to about 25%, or from about 25% to about 30% of NAC, NACA, NAC derivative, NAC metabolite, NAC congener, or D-NAC, or a pharmaceutically-acceptable salt thereof in a dry powder formulation. In some embodiments, a pharmaceutical composition of the disclosure can comprise about 5%, about 10%, about 15%, about 20%, about 25%, or about 30% of NAC, NACA, NAC derivative, NAC metabolite, NAC congener, or D-NAC, or a pharmaceutically-acceptable salt thereof in a dry powder formulation. In some embodiments, a pharmaceutical composition of the disclosure can comprise about 10%, about 11%, about 12%, about 13%, about 14%, about 15%, about 16%, about 17%, about 18%, about 19%, or about 20% of NAC, NACA, NAC derivative, NAC metabolite, NAC congener, or D-NAC, or a pharmaceutically-acceptable salt thereof in a dry powder formulation. In some embodiments, a pharmaceutical composition of the disclosure can comprise about 15% of NAC, NACA, NAC derivative, NAC metabolite, NAC congener, or D-NAC, or a pharmaceutically-acceptable salt thereof in an aqueous solution. In some embodiments, a pharmaceutical composition of the disclosure can comprise about 20% of NAC, NACA, NAC derivative, NAC metabolite, NAC congener, or D-NAC, or a pharmaceutically-acceptable salt thereof in a dry powder formulation. In some embodiments, a pharmaceutical composition of the disclosure can comprise about 25% of NAC, NACA, NAC derivative, NAC metabolite, NAC congener, or D-NAC, or a pharmaceutically-acceptable salt thereof in a dry powder formulation.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the invention is administered in combination with, before, or after treatment with another therapeutic agent. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 h of the onset of the symptoms, within the first 24 h of the onset of the symptoms, within the first 6 h of the onset of the symptoms, or within 3 h of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

A compound or pharmaceutical composition of the disclosure can be administered more than one time. In some embodiments, a compound or pharmaceutical composition of the disclosure can be administered once daily. In some embodiments, a compound or pharmaceutical composition of the disclosure can be administered twice daily. In some embodiments, a compound or pharmaceutical composition of the disclosure can be administered three times daily. In some embodiments, a compound or pharmaceutical composition of the disclosure can be administered, and the administration can be repeated at least once. In some embodiments, administration of a compound or a pharmaceutical composition can be repeated once. In some embodiments, administration of a compound or a pharmaceutical composition can be repeated twice. In some embodiments, administration of a compound or a pharmaceutical composition can be repeated three times.

In some embodiments, administration of a compound or a pharmaceutical composition can be repeated after about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, administration of a compound or pharmaceutical composition can be repeated after about 7 days. In some embodiments, administration of a compound or pharmaceutical composition can be repeated after about 14 days.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing

Compounds or pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A dose can be expressed in terms of an amount of the drug divided by the mass of the subject, for example, milligrams of drug per kilograms of subject body mass. A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg. In some embodiments, a method of the disclosure administers a therapeutically-effective amount from about 100 mg to about 400 mg.

In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg. In some embodiments, a compound described herein can be present in a composition in a range of from about 20 mg/kg to about 400 mg/kg. In some embodiments, a compound described herein can be present in a composition in a range of from about 20 mg/kg to about 240 mg/kg. In some embodiments, a compound described herein can be present in a composition in a range of from about 75 mg/kg to about 150 mg/kg. In some embodiments, a compound described herein can be present in a composition in a range of from about 75 mg/kg to about 150 mg/kg. In some embodiments, a compound described herein can be present in a composition in a range of from about 100 mg/kg to about 150 mg/kg.

In some embodiments, a compound described herein can be present in a composition in an amount of about 75 mg/kg. In some embodiments, a compound described herein can be present in a composition in an amount of about 100 mg/kg. In some embodiments, a compound described herein can be present in a composition in an amount of about 150 mg/kg. In some embodiments, a compound described herein can be present in a composition in an amount of about 200 mg/kg. In some embodiments, a compound described herein can be present in a composition in an amount of about 250 mg/kg. In some embodiments, a compound described herein can be present in a composition in an amount of about 400 mg/kg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a compound described herein can be present in a composition in an amount of about 100 mg, about 120 mg, about 140 mg, about 160 mg, about 180 mg, about 200 mg, about 220 mg, about 240 mg, about 260 mg, about 280 mg, or about 300 mg. In some embodiments, a compound described herein can be present in a composition in an amount of about 150 mg. In some embodiments, a compound described herein can be present in a composition in an amount of about 170 mg. In some embodiments, a compound described herein can be present in a composition in an amount of about 280 mg. In some embodiments, a compound described herein can be present in a composition in an amount of about 300 mg. In some embodiments, a compound described herein can be present in a composition in an amount of about 350 mg. In some embodiments, a compound described herein can be present in a composition in an amount of about 400 mg.

### Combination Therapy

The compounds or pharmaceutical compositions of the disclosure can be administered with at least one additional therapeutic agent. In some embodiments, the compounds or pharmaceutical compositions of the disclosure can be administered with one additional therapeutic agent. In some embodiments, the compounds or pharmaceutical compositions of the disclosure can be administered with two additional therapeutic agents. In some embodiments, the compounds or pharmaceutical compositions of the disclosure can be administered with three additional therapeutic agents.

In some embodiments, the therapeutic agent is a 5-lipogenase-activating protein (FLAP) inhibitor. In some embodiments, the FLAP inhibitor is MK-866 (L 663536), quiflapon (MK-591), fiboflapon (GSK2190915; AM-803), veliflapon (BAY X 1005; DG-031), AM679, or a pharmaceutically-acceptable salt thereof. In some embodiments, the therapeutic agent is glutathione. In some embodiments, the therapeutic agent is a glutathione-decorated nanoparticle.

In some embodiments, the therapeutic agent is a Cathepsin B inhibitor. In some embodiments, the Cathepsin B inhibitor is antipain dihydrochloride, CA-074, CA-074 methyl ester, Calpain inhibitor I, Calpain inhibitor II, chymostatin, cystatin, E-64, leupeptin trifluoroacetate salt, procathepsin B fragment, Z-Leu-Leu-Leu fluoromethyl ketone. In some embodiments, the Cathepsin B inhibitor is antipain dihydrochloride. In some embodiments, the Cathepsin B inhibitor is CA-074. In some embodiments, the Cathepsin B inhibitor is cystatin. In some embodiments, the Cathepsin B inhibitor is chymostatin.

In some embodiments, the therapeutic agent is a poly(ADP-ribose) polymerase (PARP) inhibitor. In some embodiments, the PARP inhibitor is olaparib, rucaparib, niraparib, talazoparib, veliparib, pamiparib, rucaparib, CEP 9722, E7016, Iniparib, or 3-aminobenzamide. In some embodiments, the PARP inhibitor is olaparib. In some embodiments, the PARP inhibitor is rucaparib. In some embodiments, the PARP inhibitor is niraparib. In some embodiments, the PARP inhibitor istalazoparib.

In some embodiments, the therapeutic agent is probenecid. In some embodiments, the therapeutic agent is phenserine. In some embodiments, the therapeutic agent is a dopaminergic agent.

### Method of Treatment

The present disclosure describes the use of a compound to treat a brain condition. In some embodiments, the brain condition is a neurological disorder. A neurological disorder is any disorder of the nervous system. Structural, biochemical, or electrical abnormalities in the brain, spinal cord, or other nerves can result in a range of symptoms. Examples of symptoms that arise from neurological disorders include paralysis, muscle weakness, poor coordination, loss of sensation, seizures, confusion, pain, and altered levels of consciousness. In some embodiments, the disclosure describes the use of a compound to treat brain damage, such as cerebral lobe (e.g., basal ganglia, cerebellum, or the brainstem) damage, frontal lobe damage, parietal lobe damage, temporal lobe damage, or occipital lobe damage. In some embodiments, the present disclosure describes the use of a compound to treat brain dysfunction according to type: aphasia (language), dysgraphia (writing), dysarthria (speech), apraxia (patterns of sequences of movements), agnosia (identifying things or people), or amnesia (memory). In some embodiments the present disclosure describes the use of a compound to treat spinal cord disorders, peripheral neuropathy and other peripheral nervous system disorders, cranial nerve disorders (e.g., Trigeminal neuralgia), autonomic nervous system disorders (e.g., dysautonomia, Multiple System Atrophy), or seizure disorders (i.e., epilepsy).

In some embodiments, the brain condition is a movement disorder of the central and peripheral nervous system, such as essential tremor, amyotrophic lateral sclerosis (ALS), Tourette's syndrome, multiple sclerosis, Parkinson's disease, or peripheral neuropathy. In some embodiments, the movement disorder is Parkinson's disease. In some embodiments, the disclosure describes the use of a compound to treat sleep disorders (e.g., narcolepsy), migraines and other types of headaches, or central neuropathy. In some embodiments, the disclosure describes the use of a compound to treat a neuropsychiatric illness, such as attention deficit hyperactivity disorder, autism, or obsessive compulsive disorder.

In some embodiments, the brain condition is a CNS condition. CNS disorders are a group of neurological disorders that affect the structure or function of the brain or spinal cord, which collectively form the CNS. The disclosure describes use of a compound to treat a CNS disorder caused by traumatic brain injury, concussion, post-concussion syndrome, infections, degeneration (e.g., degenerative spinal disorders), structural defects (e.g., anencephaly, hypospadias, spina bifida, microgyria, polymicrogyria, bilateral frontoparietal polymicrogyria, or pachgyria), tumors, autoimmune disorders, or stroke. In some embodiments, the disclosure describes the use of a compound to treat traumatic brain injury. In some embodiments, the disclosure describes the use of a compound to treat subarachnoid hemorrhage. In some embodiments, the disclosure describes the use of a compound to treat concussion. In some embodiments, the disclosure describes the use of a compound to treat post-concussion syndrome.

In some embodiments, the disclosure describes the use of a compound to treat stroke. Stroke is a medical condition in which poor blood flow to the brain results in cell death. The two main types of strokes are ischemic stroke resulting from a lack of blood flow, and hemorrhagic stroke resulting from bleeding. Signs and symptoms of a stroke may include an inability to move or feel on one side of the body, problems understanding or speaking, and a loss of vision to one side. In some embodiments, the disclosure describes the use of a compound to treat hemorrhagic stroke. In some embodiments, the disclosure describes the use of a compound to treat ICH stroke.

In some embodiments, a compound of the disclosure can be used to treat brain dysfunction. In some embodiments, a compound of the disclosure can be used to treat aphasia (language), dysgraphia (writing), dysarthria (speech), apraxia (patterns of sequences or movements), agnosia (identifying things or people), or amnesia (memory). In some embodiments, a compound of the disclosure can be used to treat a spinal cord disorder, peripheral neuropathy, a peripheral nervous system disorder, cranial nerve disorder, autonomic nervous system disorder, or a seizure disorder. In some embodiments, a compound of the disclosure can be used to treat a cranial nerve disorder, for example, trigeminal neuralgia. In some embodiments, a compound of the disclosure can be used to treat an autonomic nervous system disorder, for example, dysautonomia or multiple system atrophy. In some embodiments, a compound of the disclosure can be used to treat a seizure disorder, for example, epilepsy.

In some embodiments, the disclosure describes the use of a compound to treat brain cancer. In some embodiments, the brain cancer is an astrocytoma of the brain or spinal cord. In some embodiments, the brain cancer is a brain stem glioma. In some embodiments, the brain cancer is glioblastoma multiforme. In some embodiments, the brain cancer is meningioma. In some embodiments, the brain cancer is an ependymoma. In some embodiments, the brain cancer is an oligodendroglioma. In some embodiments, the brain cancer is a mixed glioma. In some embodiments, the brain cancer is a pituitary cancer. In some embodiments, the brain cancer is a craniopharyngioma. In some embodiments, the brain cancer is a germ cell tumor, pineal region tumor, medulloblastoma, or primary CNS lymphoma.

Administering NAC, NACA, NAC derivative, NAC metabolite, NAC congener, or D-NAC, or a pharmaceutically-acceptable salt thereof can change the concentration of a NAC neurometabolite in a brain region. In some embodiments, the administering increases the concentration of a NAC neurometabolite in the brain region. In some embodiments, the administering increased the concentration of a NAC neurometabolite in the brain region by from about 20% to about 300%. In some embodiments, the administering increased the concentration of a NAC neurometabolite in the brain region by from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. In some embodiments, the administering increased the concentration of a NAC neurometabolite in the brain region by from about 100% to about 110%, from about 110% to about 120%, from about 120% to about 140%, from about 140% to about 160%, from about 160% to about 180%, from about 180% to about 200%, from about 200% to about 220%, from about 220% to about 240%, from about 240% to about 260%, from about 260% to about 280%, or from about 280% to about 300%.

In some embodiments, the administering increased the concentration of a NAC neurometabolite in the brain region by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%. In some embodiments, the administering increased the concentration of a NAC neurometabolite in the brain region by about 20%. In some embodiments, the administering increased the concentration of a NAC neurometabolite in the brain region by about 50%. In some embodiments, the administering increased the concentration of a NAC neurometabolite in the brain region by about 100%. In some embodiments, the administering increased the concentration of a NAC neurometabolite in the brain region by about 150%. In some embodiments, the administering increased the concentration of a NAC neurometabolite in the brain region by about 200%.

In some embodiments, the administering of NAC, NACA, NAC derivative, NAC metabolite, NAC congener, or D-NAC, or a pharmaceutically-acceptable salt thereof modulates the NAC neurometabolite/water ratio in a brain region. In some embodiments, the administering of NAC, NACA, NAC derivative, NAC metabolite, NAC congener, or D-NAC, or a pharmaceutically-acceptable salt thereof modulates the GSH/water ratio in a brain region. In some embodiments, the administering of NAC, NACA, NAC derivative, NAC metabolite, NAC congener, or D-NAC, or a pharmaceutically-acceptable salt thereof increases the GSH/water ratio in a brain region. In some embodiments, the administering of NAC, NACA, NAC derivative, NAC metabolite, NAC congener, or D-NAC, or a pharmaceutically-acceptable salt thereof modulates the NAA/water ratio in a brain region. In some embodiments, the administering of NAC, NACA, NAC derivative, NAC metabolite, NAC congener, or D-NAC, or a pharmaceutically-acceptable salt thereof increases the NAA/water ratio in a brain region.

In some embodiments, the administering of NAC, NACA, NAC derivative, NAC metabolite, NAC congener, or D-NAC, or a pharmaceutically-acceptable salt thereof increases the GSH/water ratio in a region of the brain by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the administering increases the GSH/water ratio in a region of the brain by about 10%. In some embodiments, the administering increases the GSH/water ratio in a region of the brain by about 20%. In some embodiments, the administering increases the GSH/water ratio in a region of the brain by about 30%. In some embodiments, the administering of NAC, NACA, NAC derivative, NAC metabolite, NAC congener, or D-NAC, or a pharmaceutically-acceptable salt thereof decreases the GSH/water ratio in a region of the brain by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the administering decreases the GSH/water ratio in a region of the brain by about 10%. In some embodiments, the administering decreases the GSH/water ratio in a region of the brain by about 20%. In some embodiments, the administering decreases the GSH/water ratio in a region of the brain by about 30%.

In some embodiments, the administering of NAC, NACA, NAC derivative, NAC metabolite, NAC congener, or D-NAC, or a pharmaceutically-acceptable salt thereof increases the NAA/water ratio in a region of the brain by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the administering increases the NAA/water ratio in a region of the brain by about 10%. In some embodiments, the administering increases the NAA/water ratio in a region of the brain by about 20%. In some embodiments, the administering increases the NAA/water ratio in a region of the brain by about 30%. In some embodiments, the administering of NAC, NACA, NAC derivative, NAC metabolite, NAC congener, or D-NAC, or a pharmaceutically-acceptable salt thereof decreases the NAA/water ratio in a region of the brain by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the administering decreases the NAA/water ratio in a region of the brain by about 10%. In some embodiments, the administering decreases the NAA/water ratio in a region of the brain by about 20%. In some embodiments, the administering decreases the NAA/water ratio in a region of the brain by about 30%.

In some embodiments, the administering of NAC, NACA, NAC derivative, NAC metabolite, NAC congener, or D-NAC, or a pharmaceutically-acceptable salt thereof modulates the NAC neurometabolite/creatine ratio in a brain region. In some embodiments, the administering of NAC, NACA, NAC derivative, NAC metabolite, NAC congener, or D-NAC, or a pharmaceutically-acceptable salt thereof modulates the GSH/creatine ratio in a brain region. In some embodiments, the administering of NAC, NACA, NAC derivative, NAC metabolite, NAC congener, or D-NAC, or a pharmaceutically-acceptable salt thereof increases the GSH/creatine ratio in a brain region. In some embodiments, the administering of NAC, NACA, NAC derivative, NAC metabolite, NAC congener, or D-NAC, or a pharmaceutically-acceptable salt thereof modulates the NAA/creatine ratio in a brain region. In some embodiments, the administering of NAC, NACA, NAC derivative, NAC metabolite, NAC congener, or D-NAC, or a pharmaceutically-acceptable salt thereof increases the NAA/creatine ratio in a brain region.

In some embodiments, the administering of NAC, NACA, NAC derivative, NAC metabolite, NAC congener, or D-NAC, or a pharmaceutically-acceptable salt thereof increases the GSH/creatine ratio in a region of the brain by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the administering increases the GSH/creatine ratio in a region of the brain by about 10%. In some embodiments, the administering increases the GSH/creatine ratio in a region of the brain by about 20%. In some embodiments, the administering increases the GSH/creatine ratio in a region of the brain by about 30%. In some embodiments, the administering of NAC, NACA, NAC derivative, NAC metabolite, NAC congener, or D-NAC, or a pharmaceutically-acceptable salt thereof decreases the GSH/creatine ratio in a region of the brain by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the administering decreases the GSH/creatine ratio in a region of the brain by about 10%. In some embodiments, the administering decreases the GSH/creatine ratio in a region of the brain by about 20%. In some embodiments, the administering decreases the GSH/creatine ratio in a region of the brain by about 30%.

In some embodiments, the administering of NAC, NACA, NAC derivative, NAC metabolite, NAC congener, or D-NAC, or a pharmaceutically-acceptable salt thereof increases the NAA/creatine ratio in a region of the brain by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the administering increases the NAA/creatine ratio in a region of the brain by about 10%. In some embodiments, the administering increases the NAA/creatine ratio in a region of the brain by about 20%. In some embodiments, the administering increases the NAA/creatine ratio in a region of the brain by about 30%. In some embodiments, the administering of NAC, NACA, NAC derivative, NAC metabolite, NAC congener, or D-NAC, or a pharmaceutically-acceptable salt thereof decreases the NAA/creatine ratio in a region of the brain by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the administering decreases the NAA/creatine ratio in a region of the brain by about 10%. In some embodiments, the administering decreases the NAA/creatine ratio in a region of the brain by about 20%. In some embodiments, the administering decreases the NAA/creatine ratio in a region of the brain by about 30%.

In some embodiments, administering NAC, NACA, NAC derivative, NAC metabolite, NAC congener, or D-NAC, or a pharmaceutically-acceptable salt thereof can increase a GSH/creatine ration and decrease an NAA/creatine ratio in a region of the brain. In some embodiments, administering NAC, NACA, NAC derivative, NAC metabolite, NAC congener, or D-NAC, or a pharmaceutically-acceptable salt thereof can increase a GSH/creatine ration by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%; and decrease an NAA/creatine ratio by about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% in a region of the brain.

### EXAMPLES

### Example 1: Phase I Study of Brain Bioavailability and Safety of Intranasal NAC

A single site, single-blind, and open label six-part Phase 1 study in healthy volunteers is conducted to assess the brain bioavailability, safety, and tolerability of IN NAC utilizing MRS measurement of NAC-derived neurometabolites. Brain bioavailability of NAC-derived neurometabolites is assessed for three doses of IN NAC and compared to the effects of IN GSH. Additionally, the effect of different formulations, dosing devices, and positioning during IN administration are evaluated. Comparative brain bioavailability of NAC is measured following administration of NAC via IN, IV, or oral administration. The effects of 7-day repeat dosing of NAC via IN, IV, or oral administration are determined using ¹H-MRS.

Measurements are obtained pre-dose and post-dose ¹H-MRS of NAC-derived brain metabolites during single ascending and repeat dose studies of IN NAC. Blood levels of NAC (free and total), cysteine, GSH and GSH/GSSG ratios are also obtained, and measurements of cerebrospinal fluid (CSF) NAC are obtained before and after 7 days of repeat dosing. Safety and tolerability are assessed through reports of adverse events, findings on physical neurologic examinations, laboratory test results, findings on the electrocardiogram (ECG), and specific assessment for nasal tolerability.

For each part of the study, participants undergo screening beginning up to 28 days prior to IP administration on Day 1. Subjects are required to sign an informed consent form (ICF) before undertaking any study-specific procedures or assessments. Participants who qualify for the study based on inclusion and exclusion criteria are enrolled. In each part of the study, safety is monitored by assessment of adverse events (AEs), vulnerability assessment scoring tool (VAS-T), and modified total nasal symptom (TNSS-M) scores, electrocardiogram (ECG) results, vital signs, physical and neurological examinations, blood tests and urine tests.

The pharmacokinetic (PK) and pharmacodynamic (PK) properties of a single dose of IN GSH and single ascending doses of IN NAC are determined by measuring the effect of the doses on NAC-derived neurometabolites as assessed by 1) ¹H-MRS measurements of the N-cysteinyl resonances of GSH and NAC and the N-acetyl resonances of NAC and N-acetyl aspartate (NAA), expressed as ratios to the water or creatine resonance of voxels in the dorsolateral prefrontal cortex (DLPF), occipital lobe and striatum; and 2) peripheral blood concentrations of GSH, free and total NAC, cysteine and RBC GSH/GSSH ratios. In each part of the study, MRS analysis is completed and PK samples are drawn pre-dose and at 1, 3, 6 and 24 hours post-dose.

The effects of IN NAC administered under different dosing conditions, formulations, using different devices, and participant positioning during IP administration are determined by measuring NAC-derived neurometabolites in the voxels of interest using MRS. The effects of twice-daily dosing of IN NAC for 7 days on MRS-determined levels of 1) the aforementioned NAC-derived neurometabolites in the voxels of interest, 2) CSF NAC levels, and 3) peripheral blood concentrations of GSH, free and total NAC, cysteine and RBC GSH/GSSH ratios are measured.

Participant population: The study is conducted in 72 healthy male and female volunteers, inclusive at the time of informed consent. Women of childbearing potential (WOCBP) may be included and are subject to contraceptive requirements during the study from screening until study completion, including the follow-up period, and for at least 90 days after the last dose of IP. WOCBP must demonstrate negative pregnancy testing at screening and before administration of IP. The maximum duration of involvement for each participant, screening through study completion, is approximately 64-78 days.

Inclusion criteria: 1) Healthy volunteers between 18 and 45 years of age inclusive at the time of informed consent; 2) In good general health as determined by medical history, physical examination, vital signs, laboratory tests, and ECG. Isolated out-of-range values judged by the Principal Investigator (PI) or designated physician to be of no clinical significance can be allowed: the rationale for this determination must be recorded in the participant's source documents; 3) Have a body weight in the range of 50 to 120 kg, inclusive, and a body mass index (BMI) of 19 to 28 kg/m², inclusive, at screening; 4) Agree to abstain from alcohol intake for 24 hours prior to IP administration and 24 hours prior to all other outpatient clinic visits; 5) Agree not to use prescription medications (except for birth control) within 14 days prior to IP administration and for the duration of the study, unless approved by the PI and Sponsor Medical Monitor; 6) Agree not to use over the counter (OTC) medications (including corticosteroids, aspirin, pain medications, decongestants, antihistamines) and herbal medication (including St. John's Wort) within 14 days prior to IP administration through to the Day 7 follow-up visit, unless approved by the Medical Monitor. Occasional use of paracetamol (up to 2 g/day) is permitted; 7) Agree to refrain from participation in a competitive collision sport from the initiation of the screening period to the Day 28 follow up; 8) WOCBP must be non-pregnant and must use an acceptable, highly effective double barrier contraception from Screening until study completion, including the follow-up period. Double barrier contraception is defined as use of a condom (male or female, by self-declaration) AND one form of the following: established hormonal contraception (e.g., oral contraceptives pills [OCPs], long-acting implantable hormones, injectable hormones); a vaginal ring or an intrauterine device [IUD]); documented evidence of surgical sterilization at least 6 months prior to Screening (e.g., tubal occlusion, hysterectomy, bilateral salpingectomy, or bilateral oophorectomy); WOCBP who are in same-sex or not in any sexual relations (abstinence from heterosexual intercourse, by self-declaration) are not required to use contraception when this is their preferred and usual lifestyle. These WOCBP must agree to use the aforementioned acceptable, highly effective contraceptive method if they begin or plan to begin heterosexual relations from Screening until 90 days after the last dose of study drug. WOCBP must have a negative pregnancy test at Screening and Day −1 and be willing to have additional pregnancy tests as required throughout the study.

Women not of childbearing potential (non-WOCBP) must be postmenopausal for >12 months. Postmenopausal status is be confirmed through testing of follicle-stimulating hormone (FSH) levels ≥40 IU/mL at Screening for amenorrhoeic female participants. Non-WOCBP are not required to use contraception. Periodic abstinence (e.g., calendar, ovulation, symptothermal, post-ovulation-methods) and withdrawal are not considered highly effective methods of birth control. Male participants engaged in sexual relations with WOCBP must use an acceptable, highly effective double barrier contraceptive method from Screening until at least 90 days after the last dosing of study drug. Double barrier contraception is defined as use of a condom (male or female, by self-declaration) and, for WOCBP, use (by self-declaration) of an effective contraceptive including OCPs, long-acting implantable hormones, injectable hormones, a vaginal ring or an IUD (by self-declaration) or having received surgical sterilization (e.g., tubal occlusion, hysterectomy, bilateral salpingectomy, or bilateral oophorectomy).

Men in same-sex or not in any sexual relations (abstinence from heterosexual intercourse, by self-declaration) are not required to use contraception if this is their preferred and usual lifestyle. These men must agree to use the aforementioned acceptable, highly effective contraceptive method if they begin or plan to begin heterosexual relations with WOCBP from Screening until 90 days after the last dosing of study drug.

Exclusion criteria: 1) Females who are pregnant or nursing at Screening; 2) Have a deformity of the nasal cavity, a known deviation of the nasal septum; acute or chronic sinusitis or recent (<5 years) history of surgery of the nasal cavity and/or nasopharynx; 3) History of seizures or epilepsy within the past 5 years; 4) History of moderate to severe traumatic brain injury; 5) History of concussion within the past 1 year; 6) Currently have or have a history of any clinically significant medical illness ormedical disorders the Investigator considers should exclude the participant, including (but not limited to) cardiovascular, neurologic, musculoskeletal, hematologic, respiratory, dermatologic, hepatic or neoplastic disease or immune deficiency state; 7) Psychiatric or behavioral condition which would compromise participation in the study; 8) Acute upper respiratory illness including a common cold, within 14 days prior to IP administration or have had a major illness or hospitalization within 1 month of Screening; 9) Major surgery within 12 weeks of Screening; 10) Any participant who plans to undergo elective surgery within 4 weeks prior to IP administration and through the end of the study including the follow-up period; 11) Positive serology test for HIV antibodies, hepatitis B surface antigen (HBsAg), or hepatitis C virus (HCV) antibodies at Screening; 12) Recent history (within previous 6 months) of alcohol or drug abuse; 13) Have smoked tobacco or related products within 3 months prior to dosing; 14) Have positive urine drug test at Screening and/or at any time during the study for substances of abuse including but not limited to cocaine, cannabinoids, amphetamines, benzodiazepines, opiates, tricyclic antidepressants, and methadone. Participants can be re-screened once following a positive result at the discretion of the Investigator; 15) Have a positive alcohol breath test at Screening and/or at any time during the study. Patients are required to abstain from alcohol for at least 24 hours prior to IP administration on Day 1 and on the day of study assessments; 16) Consume, on average, more than approximately 500 mg/day of caffeine (as contained in 5 cups of tea or coffee or 8 cans of soda or other caffeinated products) per day; 17) Donated blood within 60 days prior to Screening; 18) Have a history of active drug and/or food allergy or other active allergic disease requiring the constant use of medications, or a history of severe allergic reaction, angioedema or anaphylaxis; 19) Received any other experimental therapy including device or an investigational agent within 30 days or 5 half-lives (whichever is longer) of IP administration; 20) Are unable to undergo MRI scanning due to the presence of non-removable metal implants, including but not limited to surgical staples, pacemaker, steel IUD etc., claustrophobia or any other contraindication.

### Statistical Methods

Pharmacokinetics: Changes in MRS spectra of the targeted metabolites and pharmacokinetic assessments (free and total NAC, cysteine and GSH concentrations and GSH/GSSG ratios and CSF NAC levels) from baseline to each post-dose timepoint are summarized using descriptive statistics.

Safety and tolerability: Participants provide a rating for IP tolerability several times during the study using a Visual Analog Scale (VAS), with a value of 0 indicating very good tolerability and 10 indicating very poor tolerability. The subjects also complete a TNSS-M, which assesses five specific nasal symptoms (i.e., congestion, runny nose, itching, pain and non-painful burning) on a 0 to 3 scale. Only items scored as a “3” (severe) on the TNSS-M are reported as adverse events.

Adverse events are coded using the most current version of the Medical Dictionary for Regulatory Activities (MedDRA®). A by-participant AE data listing, including verbatim term, preferred term (PT), system organ class (SOC), severity and relationship to IP, are provided. The number of participants experiencing treatment emergent adverse events (TEAEs), and the number of individual TEAEs are summarized by SOC, PT, and severity and relationship to IP. Laboratory evaluations, vital signs assessments, and ECG parameters are summarized for each scheduled visit. A summary of change from baseline at each protocol specified time point are presented.

Prior and concomitant medications are coded using the most current version of the World Health Organization (WHO) drug dictionary available at the start of the study and are listed by participant and summarized by treatment using anatomical therapeutic chemical (ATC) (level 2) and preferred name. Medical history, pregnancy/FSH testing, urine drug screen/alcohol breath test, physical and neurological examination, and serology (HIV, Hepatitis B and C screen) are listed by participant.

Primary objective: The primary objective of the study is to assess the brain bioavailability of intranasal (IN) NAC using proton magnetic resonance spectroscopy (¹H-MRS) assessment of change from baseline in NAC-derived metabolic markers in healthy adult volunteers

Secondary objectives: The secondary objectives of the study include: 1) Assessing the safety and tolerability of IN NAC; 2) Assessing the time course and regional CNS pharmacodynamic activity of IN NAC; 3) Comparing pharmacokinetic and pharmacodynamic activity of IN NAC to IN GSH; 4) Comparing devices and positioning during investigational product (IP) administration for optimal nose-to-brain delivery of IN NAC; 5) Assessing the regional CNS pharmacodynamic activity and safety and tolerability of IN NAC following multiple repeated IN dose; and 6) Assessing the pharmacokinetic profile of NAC in blood and cerebrospinal fluid (CSF) following IN administration.

Screen failures: Screen failures are defined as volunteers who consent to participate in the clinical study but are not subsequently enrolled. A minimal set of screen failure information is required to ensure transparent reporting of screen failure participants to meet the Consolidated Standards of Reporting Trials publishing requirements and to respond to queries from regulatory authorities. Minimal information includes screen failure details, eligibility criteria, and any serious adverse event (SAE). Individuals who do not meet the criteria for participation in this study (screen failure) are re-screened based on the judgement of the Investigator and in consultation with the medical monitor (MM). Re-screening is allowed within the recruitment period for the study. Re-screened participants are assigned the same participant number as for the initial Screening.

Participant replacement: Participants who sign the informed consent form (ICF) and are enrolled but do not receive IP may be replaced. Participants who sign the ICF, are enrolled and receive IP but subsequently withdraw, or are withdrawn or discontinued from the study, are replaced at the discretion of the Sponsor.

Participant withdrawal criteria: Participants can withdraw their consent to participate in the study at any time. If a participant withdraws consent, the date and reason for consent withdrawal are documented. Participants are encouraged to remain in the clinic to complete all necessary assessments and until the Investigator deems that it is safe to be discharged. Participant data are included in the analysis up to the date of the withdrawal of consent.

The primary reason for withdrawal is identified and recorded on the appropriate eCRF, along with the date of withdrawal. In accordance with applicable regulations, a participant has the right to withdraw from the study, at any time and for any reason, without prejudice to future medical care. If a participant is withdrawn because of an AE, the Investigator arranges for the participant to have appropriate follow-up care until the AE is resolved or has stabilized. Unresolved AEs are followed until the last scheduled Follow-up visit or until the PI and MM determine that further follow-up is no longer indicated. In addition to AEs, other reasons for removal of participants from the study can include, but are not limited to, withdrawal of consent, administrative decision by the Investigator or the Sponsor, protocol deviation, or participant noncompliance.

If a participant asks or decides to withdraw from the study, all efforts are made to complete and report the observations, especially the listed primary and secondary objectives, as thoroughly as possible up to the date of withdrawal. Wherever possible, the tests and evaluations, including those listed for the Follow-up Visit, are performed for all participants who discontinue prior to the completion of the study.

Participant termination criteria: Reasons for early termination of individual participants can include: Protocol deviations or participant non-compliance (must be specified on the appropriate electronic case report form [eCRF]); Pregnancy; Serious or severe AEs; Administrative decision by the Investigator or the Sponsor; Death; or Other (must be specified).

Lost to follow-up: A participant is considered lost to follow-up if they fail to return for one of the scheduled visits and is unable to be contacted by the study staff. The following actions are taken if a participant fails to return for a required study visit: The site attempts to contact the participant and reschedule the missed visit within 2 days and counsel the participant on the importance of maintaining the assigned visit schedule and ascertain if the participant wishes to continue in the study; Before a participant is deemed lost to follow-up, the Investigator or designee makes every effort to regain contact with the participant (three telephone calls and contact via email and text message). These contact attempts are documented in the participant's medical record or study file. The participant is considered to have withdrawn from the study with a primary reason of lost to follow-up if the staff cannot contact the participant.

### Example 2: Investigational Product, Dosage, and Mode of Administration

Participants receive one or more of IP formulation and dosage of NAC. A dose of IN NAC yielding an increase in brain GSH of approximately 13% is considered the minimal effective dose.

Intranasal NAC: For IN administration of NAC, a 20% aqueous NAC solution for inhalation or equivalent is administered intranasally at the following doses: i) 100 mg (0.5 mL)—approximately 0.25 mL in each nostril per dose; ii) 200 mg (1.0 mL)—approximately 0.50 mL in each nostril per dose; or iii) 400 mg (2.0 mL)—approximately 0.50 mL once in each nostril, with repeat administration after 5 minutes. The 20% NAC solution is supplied as a clear, colorless solution in 4 mL or 30 mL glass vials. The solution contains 200 mg/mL (20% w/v) acetylcysteine, with disodium edetate, sodium hydroxide and water. The product can also contain hydrochloric acid for pH adjustment. The pH is maintained in the range of 6.0-7.5. The NAC 20% solution is administered via one of two devices: a) Teleflex LMA® MAD Nasal™ Intranasal Mucosal Atomization Device; or b) Aptar CPS 5-mL Nasal Pump.

Intranasal GSH: For IN administration of GSH, a 20% aqueous GSH solution or equivalent is administered intranasally at a dose of 200 mg (1.0 mL), approximately 0.50 mL in each nostril. IN GSH is administered using a Teleflex MAD device.

Oral NAC: For oral administration of NAC, a 200 mg/mL NAC solution (20% w/v) is used. NAC for oral administration is prepared by diluting 20 mL of a 20% NAC solution in 60 mL of a diet soft drink, which provides a dose of 4,000 mg of NAC in a 5% solution.

IV NAC: An NAC 200 mg/mL solution for injection or equivalent is used for IV administration. The amount of NAC solution equivalent to 150 mg/kg NAC is diluted in 200 mL of 0.45% saline solution and is administered by IV over 1 hour. NAC (acetylcysteine) injection, 200 mg/mL is provided as a clear, colorless, sterile solution containing 20% w/v acetylcysteine in 30 mL vials. The solution also contains sodium hydroxide for pH adjustment and sterile water for injection.

Devices for administration: The IP is administered using: 1) an LMA® MAD Nasal™ Intranasal Mucosal Atomization Device; or 2) an Aptar CPS 5-mL Nasal Pump.

Investigational product storage: Upon receipt, the acetylcysteine 20% solution for inhalation is stored at controlled room temperature in an area protected from light and maintained at a temperature below 25° C. Remaining undiluted solution in opened vials are stored under refrigeration and used within 96 hours. The GSH solution is stored at controlled room temperature. Unopened IV NAC solutions are stored at controlled room temperature, and previously opened IN NAC vials are not used for IV administration.

### Example 3: Study Evaluations and Measurements

Pharmacodynamic assessments: Pharmacodynamic assessments include change from baseline in NAC-derived neurometabolite concentrations in three brain regions (i.e., occipital cortex, striatum and DLPF), using ¹H-MRS following a single dose of IN NAC in healthy volunteers at 1, 3, 6 and 24 hours post-dose.

¹H-MRS analysis is performed using 3.0 cm×3.0 cm×2.5 cm voxels placed in the left dorsal striatum at the level of the lentiform nucleus, the occipital cortex, and the dorsolateral prefrontal cortex (DLPF). A J-edited spin echo difference method is implemented with an echo time (TE) of 70 ms and a repetition time (TR) of 1500 ms using 240 interleaved excitations (480 total) for an acquisition time of 12.5 minutes per voxel. A pair of frequency-selective inversion pulses are inserted into the standard point-resolved spectroscopy method and applied on alternate scans at the frequency of the reduced form of glutathione α-cysteinyl resonance at 4.56 ppm while avoiding excitation of the oxidized form of glutathione α-cysteinyl resonance at 3.28 ppm. Subtracting the two, resulting inverted subspectra of GSH yield a ¹H-MRS only consisting of GSH β-cysteinyl resonance at 2.98 ppm. The 32-channel phased-array coil GSH data are combined into a single regular time-domain free-induction decay signal using the unsuppressed voxel tissue water signal from each receiver coil element to derive the required relative phased-array coil sensitivities. The metabolite concentrations are estimated by calculating the areas of the individual spectral peaks obtained by frequency-domain fitting each resonance to a Gauss-Lorentz lineshape function using the Levenberg-Marquardt non-linear least-squares algorithm.

Pharmacokinetic assessments: Pharmacokinetic assessments include peripheral blood measurements of GSH, cysteine, free and total NAC and reduced-to-oxidized GSH ratio (GSH/GSSG) ratios at 1, 3 6 and 24 hours following IN NAC or GSH administration, and levels of CSF NAC obtained via lumbar puncture 6 hours following IP administration in Parts 5 and 6 of the study.

Blood PK sample collection: Blood PK samples are collected as close as possible prior to the acquisition of MRS data at each time point specified in the Schedule of Assessments. Blood PK measures free and total NAC, cysteine and GSH concentrations and GSH/GSSG ratio. GSH/GSSG is measured in whole blood using high performance liquid chromatography (HPLC) coupled to a mass spectrometer (MS). Total protein-bound and total protein-unbound concentrations of NAC, Cys and GSH are measured in plasma using a validated HPLC-MS assay.

Pharmacodynamic endpoints: MRS of NAC-derived brain metabolites in three regions of interest (occipital cortex, striatum, DLPF) at baseline and at 1, 3 and 6 hours following IN NAC are summarized using descriptive statistics. Change from baseline to each post-dose measurement are summarized descriptively. No a priori inferential statistical tests are planned. Brain regions of interest and timing of MRS are modified based on initial results.

Pharmacokinetic endpoints: A descriptive summary of the quantifiable concentrations of the targeted metabolites are reported for the specified time points to assess free and total NAC, cysteine and GSH concentrations and reduced-to-oxidized GSH ratio (GSH/GSSG).

Safety and tolerability: All safety assessments, including prior and concomitant medications, AEs, laboratory evaluations, vital signs, ECGs, and other safety assessments are summarized using the Safety Population.

Prior and concomitant medication: Prior and concomitant medications are coded using the most current version of the WHO drug dictionary available at the start of the study. Prior and concomitant medications are listed by participant and summarized by treatment using ATC (level 2) and preferred name.

Adverse events: Adverse events re coded using the most current version of the MedDRA® available. A by participant AE data listing, including verbatim term, PT, SOC, severity and relationship to IP, are provided. The number of participants experiencing TEAEs, and the number of individual TEAEs are summarized by SOC, PT, severity and relationship to IP.

Other safety assessments: Other safety assessments listed by participant include: medical history, pregnancy test, urine drug screen, alcohol breath test, physical and neurological examination, and serology (e.g., HIV, Hepatitis B, Hepatitis C).

Safety parameters: Study procedures are completed as delineated in the Schedule of Assessments. If a participant is unable to attend a visit within the specified window, the Investigator or designee discusses appropriate scheduling with the Sponsor's MM or appropriate designee. Any unscheduled procedures required for urgent evaluation of safety concerns take precedence over all routine scheduled procedures.

Demographic and medical history: Medical history (e.g., concomitant mediations, alcohol and smoking status, and drug use), date of birth, age (calculated), sex, ethnicity, and race are recorded at Screening.

Vital signs: Vital signs (e.g., blood pressure [systolic and diastolic], pulse rate, respiratory rate, and body temperature) are listed and summarized at protocol specified collection time point. Observed and change from baseline are summarized at each protocol specified collection time point. When the time of vital signs measurement coincides with a blood draw, the vital signs are taken before the scheduled blood draw where possible, ensuring the blood draw is within the window specified in the protocol. Additional vital signs are performed at other times if deemed necessary.

Weight and height: Body height and body weight are measured at Screening and are used to calculate BMI. BMI is calculated by dividing the participant's body weight in kilograms by the participant's height in meters squared (kg/m2). Body weight and height are obtained with the participant's shoes and jacket or coat removed.

Physical and neurological examination: Full and brief physical and neurological examinations are performed by a licensed physician at the time points specified in the Schedule of Assessments. Full physical examinations include: general appearance, head, ears, eyes, nose, throat, dentition, thyroid, chest (heart, lungs), abdomen, skin, neurological, extremities, back, neck, musculoskeletal, and lymph nodes. The neurological examination includes assessment of mental status and function of cranial nerves, motor and sensory systems, gait/coordination and deep tendon reflexes. Brief physical examination includes: head, ears, eyes, nose, throat, chest (heart, lungs), abdomen, skin, musculoskeletal, and lymph nodes and any pertinent system based on any prior findings. Brief neurologic examination includes assessment of eye movements, facial symmetry, drift of upper extremities, coordination (finger-to-nose and heel-toe testing) and deep tendon reflexes. Physical and neurological examinations are performed at various unscheduled time points if deemed necessary by the Investigator.

Tolerability assessments: Participants provide a rating for IP tolerability at specified times during the study using VAS-T with a value of 0 indicating very good tolerability and 10 indicating very poor tolerability. Participants also complete TNSS-M, which assesses five specific nasal symptoms (congestion, runny nose, itching, pain and non-painful burning) on a 0 to 3 scale. Only items scored as a “3” (severe) on the TNSS-M are reported as adverse events.

Electrocardiograms: ECG values are listed and summarized at protocol specified collection time point. Observed and change from baseline are summarized at each protocol specified collection time point. A 12-lead ECG are taken at the time points delineated in the Schedule of Assessments. Additional ECG monitoring are performed at other times if deemed necessary. ECGs are performed prior to vital signs with participants in a supine position. Participants are in supine position for at least 5 minutes before the reading is taken. All ECG tracings are reviewed by the PI or designee. When the time of ECG monitoring coincides with a blood draw, the ECG is taken before the scheduled blood draw while ensuring the blood draw is within the window specified in the protocol.

Laboratory evaluations: Laboratory evaluations, including hematology, serum chemistry and urinalysis, are listed and summarized at each protocol specified collection time point. Observed and change from baseline clinical laboratory data are summarized at each protocol specified collection time point. A blood sample for safety laboratory testing (hematology, serum chemistry, and urinalysis) are taken at the time points specified in the Schedule of Assessments. Additional clinical laboratory tests are performed at other times if deemed necessary based on the participant's clinical condition.

Hematology parameters tested are: hemoglobin (HGB); hematocrit (HCT); erythrocytes (RBC); platelets (PLAT); leukocytes with differential, including Eosinophils (ESN), Neutrophils (NEUT), Basophils (BASO), Lymphocytes (LYM), and Monocytes (MONO). Serum chemistry parameters tested are: urea (BUN); creatinine (CREAT); total Bilirubin (BILI) and Direct Bilirubin (BILIDIR); urate (URATE); albumin (ALB); globulin (GLOBUL); alkaline Phosphatase (ALP); creatine Kinase (CK); aspartate Aminotransferase (AST); alanine Aminotransferase (ALT); gamma-GT (GGT); glucose (GLU); sodium (NA); potassium (K); calcium (CA); chloride (CL); phosphate (PHOS); bicarbonate (BICARB); and lactate dehydrogenase (LDH).

Urinalysis: A urinalysis test (dipstick) is performed for each participant. Urinary analysis is performed at Screening. If abnormality is noted for protein, blood, nitrite or leukocyte esterase (and at the discretion of the Investigator), a microscopic examination of red blood cells, white blood cells, bacteria and casts are performed. Macroscopic urinalysis parameters to be tested are: pH (PH); specific gravity (SPGRAV); creatinine (CREATININE); protein (PROT); glucose (GLUC); ketones (KETONES); total Bilirubin (BILI); occult Blood (OCCBLD); nitrite (NITRITE); urobilinogen (UROBIL); and leukocytes (WBC).

Viral serology: HBsAg, anti-HCV and HIV antibody testing are performed at Screening.

Urine drug screen and alcohol breath test: A urine drug screen is performed at Screening, prior to dosing on Day 1, and at the Day 7 Follow-up Visit. The urine drug screen includes but is not limited to cocaine, cannabinoids, amphetamines, benzodiazepines, opiates, tricyclic antidepressants and methadone. An alcohol breath test is performed at Screening, prior to dosing on Day 1, and at the Day 7 Follow-up Visit.

Pregnancy testing and follicle-stimulating hormone testing: A serum pregnancy test is performed at the Screening visit for WOCBP only. A urine pregnancy test is performed prior to dosing on Day 1. If the result is positive, a serum test is performed for confirmation. Women not of childbearing potential must be postmenopausal (defined as cessation of regular menstrual periods for at least 12 months). Postmenopausal status is confirmed through testing of FSH levels ≥40 IU/mL at Screening.

Adverse and serious adverse events: AEs are reported for all participants from the time of consent until the completion of the Follow-up Visit. Serious adverse events are reported for all participants (enrolled and not enrolled) from the time of consent until the completion of the Follow-up Visit. Adverse events reported from the time of consent up until dosing are recorded as pre-treatment AEs. Treatment-emergent AEs (TEAEs) are evaluated from the first administration of IP until the Follow-up Visit or up to a 30-day follow-up period for AEs deemed related to treatment. Adverse events that are ongoing at the final follow-up are marked as Not Recovered/Not resolved on the AE eCRF page. All spontaneously volunteered and enquired for, as well as observed AEs, are recorded in the participant's medical records and the eCRF.

An AE is any event, side-effect, or other untoward medical occurrence that occurs in conjunction with the use of a medicinal product in humans, whether or not considered to have a causal relationship to this treatment. An AE can be any unfavorable and unintended sign that can include a clinically significant abnormal laboratory finding, symptom, or disease temporally associated with the use of a medicinal product, whether or not considered related to the medicinal product.

Events meeting the definition of an AE include: 1) exacerbation of a chronic or intermittent pre-existing condition including either an increase in frequency and/or intensity of the condition; 2) new conditions detected or diagnosed after IP administration that occur during the reporting periods, even though it may have been present prior to the start of the study; 3) signs, symptoms, or the clinical sequelae of a suspected interaction; and 4) signs, symptoms, or the clinical sequelae of a suspected overdose of either IP or concomitant medications (overdose per se is be reported as an AE/SAE)

Events that do not meet the definition of an AE include: 1) Medical or surgical procedure (e.g., endoscopy, appendectomy); the condition that leads to the procedure is reported as an AE if it meets the criteria of an AE; 2) situations where an untoward medical occurrence did not occur (e.g., social and/or convenience admission to a hospital); and 3) anticipated day-to-day fluctuations of pre-existing disease(s) or condition(s) present or detected at the start of the study that do not worsen. If there is evidence of an AE through report or observation, the Investigator or designee evaluates further and record the following information: time of onset and resolution; severity; seriousness; causality/relation to study treatment; action taken regarding IP; action taken regarding AE; and outcome. Only items scored as a “3” (severe) on the TNSS-M are reported as adverse events.

Severity of an adverse event: Severity of AEs is graded by the Investigator as one of: 1) Mild (Grade 1): A type of AE that is usually transient and may require only minimal treatment or therapeutic intervention. The event does not generally interfere with usual activities of daily living; 2) Moderate (Grade 2): A type of AE that is usually alleviated with additional specific therapeutic intervention. The event interferes with usual activities of daily living, causing discomfort but poses no significant or permanent risk of harm to the research participant; 3) Severe (Grade 3): A type of AE that interrupts usual activities of daily living, or significantly affects clinical status, or may require intensive therapeutic intervention; 4) Life-threatening (Grade 4): A type of AE that places the participant at immediate risk of death; and 5) Death (Grade 5): Events that result in death.

Causal relationship of an adverse event: The Investigator assesses the relationship between IP and the occurrence of each AE. The Investigator's assessment of the relationship of each AE to IP is recorded in the source documents and the eCRF. Alternative causes, such as medical history, concomitant therapy, other risk factors, and the temporal relationship of the event to the IP is considered and investigated, if appropriate. The following definitions are general guidelines to help assign grade of attribution: 1) Not related: The event is clearly related to other factors such as the participant's environment or clinical state, therapeutic interventions or concomitant drugs administered to the participant. This is especially so when an event occurs prior to the commencement of treatment with the IP; 2) Unlikely: The temporal association, participant history, and/or circumstances are such that the IP is not likely to have had an association with the observed event. Other conditions, including concurrent illness, progression, or expression of the disease state, or reaction to a concomitant drug administered appear to explain the event; 3) Possible: The event follows a reasonable temporal sequence from the time of IP administration or follows a known response to the IP but could have been produced by other factors such as the participant's clinical state, other therapeutic interventions, or concomitant drugs administered to the participant; 4) Probable: The event follows a reasonable temporal sequence from the time of IP administration and follows a known response to the IP and cannot be reasonably explained by other factors such as the participant's clinical state, other therapeutic interventions, or concomitant drugs administered to the participant; and 5) Definite: The event follows a reasonable temporal sequence from the time of IP administration or control abates upon discontinuation or cannot be explained by known characteristics of the participant's clinical state.

Expectedness: The MM is responsible for determining whether an AE is expected or unexpected. An AE is considered unexpected if the nature, severity, or frequency of the event is not consistent with risk information.

Outcome: Outcome of an AE is recorded on the AE eCRF as follows: recovered/resolved; recovering/resolving; recovered/resolved with sequelae; not recovered/not resolving; fatal; and unknown.

Definition of serious adverse event: An SAE is an AE occurring during any study phase (i.e. baseline, treatment, or follow-up), and at any dose of the IP, that fulfils one or more of the following: results in death; it is immediately life-threatening; it requires in-patient hospitalization or prolongation of existing hospitalization; it results in persistent or significant disability or incapacity; results in a congenital abnormality or birth defect; it is an important medical event that may jeopardize the participant or may require medical intervention to prevent one of the outcomes listed above. An AE is considered “life-threatening” if, in the opinion of either the Investigator or the Sponsor, the occurrence places the participant at immediate risk of death. It does not include an AE that, had it occurred in a more severe form, might have caused death.

Notification of a serious adverse event: All SAEs are reported within 24 hours from the time the site investigational team becomes aware of the event to meet requirements for expedited reporting of SAEs to applicable regulatory authorities and institutional ethics committees. Initial reporting is achieved by completing an SAE report form and email the assigned project email address, which is provided upon study setup. If completion of an SAE form and emailing is not possible, reporting by telephone is required and a completed SAE form must be emailed at the first opportunity. Initial notification of an SAE by telephone is confirmed in writing 24 hours from the time the site investigational team first becomes aware of the event using the SAE report form as described above. As further information regarding the SAE becomes available, such follow-up information is documented on a new SAE report form, marked as a follow-up report, scanned and emailed to the address at the bottom of the report form.

Withdrawal from the study in the event of an SAE and therapeutic measures taken are at the discretion of the Investigator. A full explanation for the discontinuation from the study are made in the participant's medical records and in the CRF. The Sponsor or their designee is responsible for notifying the relevant regulatory authorities of certain events. The Investigator is also be notified of all unexpected, serious, drug-related events that occur during the clinical trial. The investigational site is responsible for notifying its IRB/EC of these additional SAEs, if required.

Clinical Laboratory Abnormalities and Other Abnormal Assessments as Adverse Events and Serious Adverse Events: Abnormal laboratory findings (e.g. serum chemistry and hematology) or other abnormal assessments (e.g. ECG and vital signs) per se are not reported as AEs. However, those abnormal findings that are deemed clinically significant by the PI and/or delegate or are associated with signs and/or symptoms are recorded as AEs if they meet the definition of an AE (and recorded as an SAE if they meet the criteria of being serious) as previously described. Clinically significant abnormal laboratory or other abnormal findings that are detected after consent or that are present at baseline and worsen after consent are included as AEs (and SAES if serious). The Investigator exercises medical and scientific judgement in deciding whether an abnormal laboratory finding, or other abnormal assessment is clinically significant. To be considered clinically significant, the abnormality is associated with a clinically evident sign or symptom or be likely to result in an evident sign or symptom in the near term. A clinically significant laboratory abnormality in the absence of clinical symptoms can jeopardize the participant and can require intervention to prevent immediate consequences. For example, a markedly low serum glucose concentration can not be accompanied by coma or convulsions yet be of a magnitude to require glucose administration to prevent such sequelae.

Recording adverse events: Adverse events spontaneously reported by the participant and/or in response to an open question from the study personnel or revealed by observation are recorded in accordance with the Investigator's normal clinical practice and on the AE page of the eCRF during the study at the investigational site. Abnormal values that constitute an SAE or lead to discontinuation of administration of IP must be reported and recorded as an AE. Information about AEs and SAES are collected from the time of consent until the end of the study. The AE term are reported in standard medical terminology when possible. For each AE, the Investigator evaluates and reports the onset (date and time), resolution (date and time), intensity, causality, action taken, serious outcome (if applicable), and whether or not it caused the participant to discontinue the study. AEs that occur during the study are documented in the participant's medical record, on the AE eCRF and on the SAE report form. If an SAE report is completed, pertinent laboratory data is recorded on the SAE form, preferably with baseline values and copies of laboratory reports.

If the abnormal assessment meets the criteria for being serious, the SAE form is also be completed. A diagnosis, if known, or clinical signs or symptoms if the diagnosis is unknown, is used to complete the AE/SAE page. If no diagnosis is known and clinical signs or symptoms are not present, then abnormal finding is recorded.

Follow-up of Adverse Events and Serious Adverse Events: All AEs and SAEs that are deemed related, possibly related or probably related to the IP are followed until resolution, until the condition stabilizes, until the event is otherwise explained, or until the participant dies or is lost to follow-up. The Investigator is responsible for ensuring that follow-up includes any supplemental investigations as may be indicated to elucidate as completely as practical the nature and/or causality of the AE/SAE. Additional laboratory tests or investigations or consultation with other health care professionals are included. The Sponsor can request that the Investigator perform or arrange for the conduct of supplemental measurements and/or evaluations. If a participant dies during participation in the study or during a recognized follow-up period, the Sponsor is provided with a copy of any post-mortem findings, including histopathology.

Pregnancy: Pregnancy testing is performed in all WOCBP at Screening and Day 1 as per the Schedule of Assessments, and the pregnancy results is captured in the eCRF. All WOCBP re instructed to contact the Investigator immediately if they suspect they might be pregnant (e.g., missed or late menstrual period) at any time during the trial. Male participants contact the Investigator immediately if they suspect they may have fathered a child during the study treatment period. When possible, the partner's pregnancy is followed (to term) to determine the outcome. Should a pregnancy occur, it must be reported and recorded on a Pregnancy Form. Pregnancy is not regarded as an AE unless there is a suspicion that the IP may have interfered with the effectiveness of a contraceptive medication. The Investigator reports the details on a Pregnancy Form to the Sponsor/assigned designee within 24 hours of knowledge of the pregnancy. Even though participants agree to withdraw or terminate the clinical trial, the Investigator follows-up and documents the process and results of all the pregnancies.

If a male participant's female partner becomes pregnant while enrolled in the trial, a Pregnancy Form is completed and sent to the Clinical Research Organization (CRO) expeditiously, irrespective of whether it meets the criteria for expedited reporting. Abortions (spontaneous, accidental, or therapeutic) are also reported. Congenital anomalies/birth defects always meet SAE criteria, and is therefore be expeditiously reported as an SAE, using the previously described process for SAE reporting. A Pregnancy Form is updated to reflect the outcome of the pregnancy. The Investigator reports any pregnancy (including pregnancy of a male participant's partner), even if no AE has occurred, on a Pregnancy Report Form within 24 hours of the Investigator becoming aware of the pregnancy

### Example 4: Single Ascending Dose Study

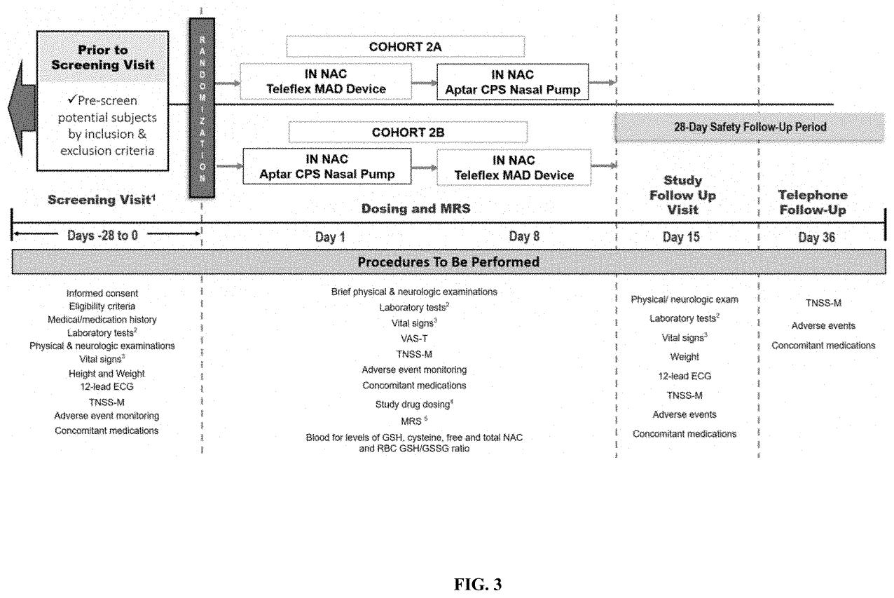

The single ascending dose study is conducted as a single-blind study with study participants blinded to treatment assignment. FIG. 2 illustrates a schematic of the single ascending dose study for intranasal administration of N-acetylcysteine. ABBREVIATIONS: ECG, electrocardiogram; GSH/GSSG, reduced-to-oxidized glutathione; MRS, magnetic resonance spectroscopy; NAC, N-acetylcysteine; TNSS-M, modified total nasal symptom scale; VAS-T, Visual analog scale for Tolerability. [1] Screening procedures must occur within 28 days of study drug administration; [2] See laboratory assessments for list of tests to be completed; [3] Vital signs include temperature, pulse, and blood pressure, and are completed before and after each MRS session; [4] IN NAC to be given immediately following pre-dose scan; [5] MRS to be completed pre-dose and 1, 3, 6, and 24 hours post-dose. The radiologist providing review and interpretation of MRS studies is blinded to the treatment assignment, IP administered, MRS timing and other details of IP administration. Twenty subjects are randomized in a 1:1 fashion to one of two dosing cohorts.

IN NAC and IN GSH are administered using a Teleflex MAD device. Study medication, IN GSH 200 mg and IN NAC 100, 200 and 400 mg, are supplied in identical-appearing nasal administration devices. The dosing procedures are identical for the NAC and GSH 200 mg doses (0.5 mL once in each nostril). The dosing procedure are 0.25 mL in each nostril for the NAC 100 mg dose, and 0.5 ml twice in each nostril for the NAC 400 mg dose. The participants receive four successive doses of study medication with intervals of one week between each dose. The subject is otherwise be blinded to treatment assignment.

Twenty healthy volunteers are randomized in a 1:1 ratio to one of two regimens consisting of either low-dose IN NAC (100 mg) or IN GSH 200 mg, followed by sequential single escalating doses of IN NAC 200 mg and IN NAC 400 mg given at 7-day intervals on Days 8, 15 and 22 (TABLE 1). Cohort 1A receives IN GSH 200 mg, followed at one week intervals by successive doses of IN NAC 100 mg, IN NAC 200 mg and IN NAC 400 mg. Cohort 1B receives IN NAC 100 mg, followed at one week intervals by successive doses of IN GSH 200 mg, IN NAC 200 mg and IN NAC 400 mg. Both NAC and GSH ARE administered as 20% aqueous solutions. On each day of study medication dosing, MRS is performed, and blood samples are collected to determine peripheral blood concentrations of GSH, cysteine, free and total NAC, and RBG GSH/GSSG ratios prior to administering study medication, and at 1-, 3-, 6-, and 24-hours afterwards.

TABLE 1

| Cohort | Dose 1 | Dose 2 | Dose 3 | Dose 4 |
|---|---|---|---|---|
| 1A | GSH | NAC | NAC | NAC |
| | 200 mg | 100 mg | 200 mg | 400 mg |
| 1B | NAC | GSH | NAC | NAC |
| | 100 mg | 200 mg | 200 mg | 400 mg |

Change from baseline in the relative levels of NAC-derived neurometabolites are assessed following administration of a single dose of IN NAC or IN GSH followed at weekly intervals by single ascending doses of IN NAC, as well as changes in peripheral blood measurements of PK laboratories.

Participants are required to remain recumbent from the baseline scan through to the final 6-hour scan to the extent possible. Safety and tolerability are monitored. as outlined in TABLE 2. If tolerability is acceptable, Participants proceed to the next planned dose of study medication. Participants experiencing treatment-limiting adverse effects are discontinued from the study and do not proceed to the next dose. Participants return for a Follow-up Visit 7 days following the last dose of IP (Day 30±3 days) and receive a follow-up telephone call on Day 51 (±2 days) for safety assessment.

TABLE 2

| | | | | | | |
|---|---|---|---|---|---|---|
| | Screening | | Doses 2, | Post- | Study | |
| | visit¹ | | 3 and 4 | Dosing | Follow-up | Telephone |
| | Day −28 | Dose 1 | Days 8, 15 | Days 2, 9, | Visit | call |
| Activity | to −1 | Day 1 | and 22 | 16 and 23 | Day 29 | Day 50 |

Screening/Administrative/Other Assessments

| | | | |
|---|---|---|---|
| Informed consent | X | | |
| Demography | X | | |
| Eligibility criteria | X | X | X |
| Medical/medica- | X | | |
| tion history | | | |
| Drug/alcohol | X | X | X |
| screen | | | |
| Laboratory tests² | X | | |
| Randomization | | X | |

Safety Assessments

| | | | | | | |
|---|---|---|---|---|---|---|
| Physical exam | X | | | | X | |
| Brief physical | | X | X | X | | |
| exam | | | | | | |
| Neurologic exam | X | | | | X | |
| Brief neurologic | | X | X | X | | |
| exam | | | | | | |
| Height | X | | | | | |
| Weight | X | | | | X | |
| 12-lead ECG | X | | | X | X | |
| Laboratory tests² | | X | X | X | X | |
| Urinalysis | X | | | | | |
| VAS-tolerability | | X | X | X | X | |
| TNSS-M | X | X | X | X | X | X |
| Adverse Event | X | X | X | X | X | X |
| Monitoring | | | | | | |
| Concomitant | X | X | X | X | X | X |
| meds | | | | | | |

IP Administration/MRS /Pharmacokinetic Assessments

| | | | |
|---|---|---|---|
| IP dosing | X | X | |
| Plasma GSH, | X | X | X |
| cysteine, free | | | |
| and total NAC³ | | | |
| RBC GSH/GSS³ | X | X | X |
| MRS⁴ | X | X | X |

ABBREVIATIONS:
ECG, electrocardiogram;
GSH, glutathione GSH/GSSG, reduced-to-oxidized glutathione;
MRS, magnetic resonance spectroscopy;
NAC, N-acetylcysteine;
TNSS-M, Modified Total Nasal Symptom Scale;
VAS-T, Visual Analog Scale for Tolerability
¹Screening procedures must occur within 28 days of Day 1 IP dosing
²See Laboratory assessments for list of tests to be completed.
³Blood samples for NAC, cysteine, GSH and RBC GSH/GSSG drawn prior to each MR session.
⁴MRS pre-dose and 1, 3, 6 and 24 hours post-dose

### Example 5: Device Comparison

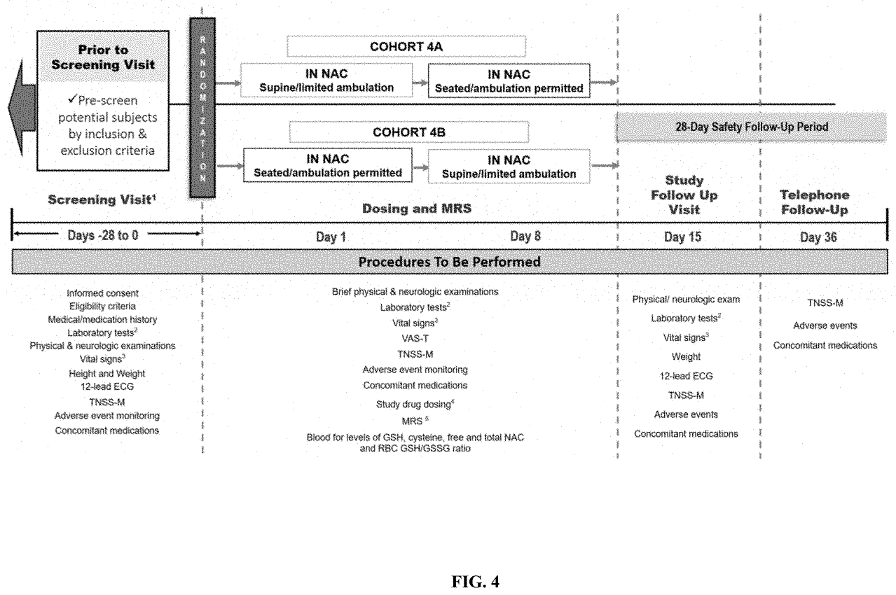

FIG. 3 illustrates a schematic for a device comparison study. ABBREVIATIONS: ECG, electrocardiogram; GSH/GSSG, reduced-to-oxidized glutathione; MRS, magnetic resonance spectroscopy; NAC, N-acetylcysteine; TNSS-M, modified total nasal symptom scale; VAS-T, Visual analog scale for Tolerability. [1] Screening procedures must occur within 28 days of study drug administration; [2] See laboratory assessments for list of tests to be completed; [3] Vital signs include temperature, pulse, and blood pressure, and are completed before and after each MRS session; [4] IN NAC to be given immediately following pre-dose scan, with dose (100, 200, or 400 mg) to be determined in single ascending dose study; [5] MRS to be completed pre-dose and 1, 3, 6, and 24 hours post-dose._Ten study participants are randomized in a 1:1 ratio to receive IN NAC (acetylcysteine 20% aqueous solution) using either a Teleflex LMA® MAD Nasal™ Intranasal Mucosal Atomization Device or Aptar CPS 5-mL Nasal Pump on Day 1. Each cohort receives IN NAC via the alternate device on Day 8. The dose of IN NAC—100, 200 or 400 mg—is selected based on the results of the single ascending dose study. TABLE 3 shows the dose cohorts used for the device comparison study.

TABLE 3

| Cohort | Day 1 | Day 8 |
|---|---|---|
| 2A | Teleflex MAD Device | Aptar CPS Nasal Pump |
| 2B | Aptar CPS Nasal Pump | Teleflex MAD Device |

MRS is performed and blood collected for determination of peripheral blood concentrations of GSH, cysteine, free and total NAC and RBG GSH/GSSG ratios prior to IP administration. MRS is repeated 1, 3, 6, and 24 hours post-dose. Changes from baseline in the relative levels of NAC-derived neurometabolites are assessed following single doses of IN NAC administered using a Teleflex LMA® MAD Nasal™ Intranasal Mucosal Atomization Device or an Aptar CPS 5-mL Nasal Pump.

IN NAC is administered as a NAC 20% solution according to the instructions for use for the respective device. Each administration can be followed by an oral rinse of approximately 200 mL of water. Safety and tolerability are monitored following the schedule of assessments of TABLE 4. Participants experiencing treatment-limiting adverse effects are discontinued from the study. Participants return for a Follow-up Visit 7 days following the last dose of the IP (Day 15±3 days) and receive a follow-up telephone call on Day 36 (±2 days) for safety assessment.

TABLE 4

| | | | | | | |
|---|---|---|---|---|---|---|
| | Screening | | Doses | Post- | Study | |
| | visit¹ | | 2 and3 | Dosing | Follow-up | Telephone |
| | Day −28 | Dose 1 | Days 8 | Days 2, 9 | Visit | Call |
| Activity | to −1 | Day 1 | and 15 | and 16 | Day 22 | Day 36 |

Screening/Administrative/Other Assessments

| | | | |
|---|---|---|---|
| Informed consent | X | | |
| Demography | X | | |
| Eligibility criteria | X | X | X |
| Medical/medica- | X | | |
| tion history | | | |
| Drug/alcohol | X | X | X |
| screen | | | |
| Laboratory tests² | X | | |
| Randomization | | X | |

Safety Assessments

| | | | | | | |
|---|---|---|---|---|---|---|
| Physical exam | X | | | | X | |
| Brief physical | | X | X | X | | |
| exam | | | | | | |
| Neurologic exam | X | | | | X | |
| Brief neurologic | | X | X | X | | |
| exam | | | | | | |
| Vital signs | X | X | X | X | X | |
| Height | X | | | | | |
| Weight | X | | | | X | |
| 12-lead ECG³ | X | | | | X | |
| Laboratory tests² | | X | X | X | X | |
| Urinalysis | X | | | | | |
| VAS-T | | X | X | X | | |
| TNSS-M | X | X | X | X | X | X |
| Adverse Event | X | X | X | X | X | X |
| Monitoring | | | | | | |
| Concomitant | X | X | X | X | X | X |
| meds | | | | | | |

IP Administration/MRS

| | | | |
|---|---|---|---|
| IP dosing | X | X | |
| MRS³ | X | X | X |

ABBREVIATIONS:
ECG, electrocardiogram;
GSH, glutathione GSH/GSSG, reduced-to-oxidized glutathione;
MRS, magnetic resonance spectroscopy;
NAC, N-acetylcysteine;
TNSS-M, Modified Total Nasal Symptom Scale;
VAS-T, Visual Analog Scale for Tolerability
¹Screening procedures must occur within 28 days of Day 1 IP dosing.
²See Laboratory assessments for list of tests to be completed.
³MRS pre-dose and 1, 3, 6 and 24 hours post-dose

### Example 6: Formulation Comparison Study

Ten study participants are randomized in a 1:1 ratio to two dosing cohorts. One cohort receives acetylcysteine 20% solution on Day 1, followed 7 days later by IV NAC, with the other cohort assigned to the reverse sequence of formulation dosing. The dose of IN NAC—100, 200 or 400 mg—and the device utilized for IP administration is selected based the results of the single ascending dose study and the dose comparison study. NAC 200 mg/mL injection or an equivalent is administered by site staff. NAC is hyperosmolar (2000 mOsm/L), so NAC is diluted prior to injection. NAC is diluted in a 0.45% saline solution (½ normal saline). The dosage of IV NAC administered is 150 mg/kg, which is diluted in 200 mL of 0.45% saline solution and infused over 1 hour. TABLE 5 lists example dosages by weight of 200 mg/mL IV NAC.

TABLE 5

| | Dose of NAC (acetylcysteine 200 |
|---|---|
| Body | mg/mL) solution for injection 150 |
| weight | mg/kg in 200 mL of 0.45% saline |
| (kg) | solution infused over 1 hour, mg |
| 50 | 7,500 |
| 60 | 9,000 |
| 70 | 10,500 |
| 80 | 12,000 |
| 90 | 13,500 |
| ≥100 | 15,000 |

MRS is performed, and blood samples are collected to determine peripheral blood concentrations of GSH, cysteine, free and total NAC and RBG GSH/GSSG ratios prior to IP administration. MRS is repeated 1, 3, 6, and 24 hours post-dose. Changes from baseline in the relative levels of NAC-derived neurometabolites are assessed following administration of single doses of IN NAC as a NAC 20% solution or IV NAC.

The 20% NAC solution is administered intranasally with the participant in a supine or seated position as instructed. During the study, a participant receives either 0.5 mL, 1 mL, or 2 mL of the 20% NAC solution or 1 mL of a 20% GSH solution. For all doses, approximately half of the total dose is administered into each nostril. Using the Teleflex MAD device, NAC doses are administrated as follows: 1) IN NAC 100 mg (0.5 mL): 0.25 mL per spray, one spray in each nostril; 2) IN NAC 200 mg (1 mL) or IN GSH 200 mg (1 mL): 0.25 mL per spray, two sprays in each nostril; or 3) IN NAC 400 mg (2 mL): 0.5 mL per spray, two sprays in each nostril, with repeat administration of two sprays in each nostril after 5 minutes. Using the Aptar CPS Nasal Pump, NAC doses are administered as follows: 1) IN NAC 100 mg (0.5 mL): 0.14 mL per spray, 2 sprays in each nostril; 2) IN NAC 200 mg (1 mL) or IN GSH 200 mg (1 mL): 0.14 mL per spray, 4 sprays in each nostril; or 3) IN NAC 400 mg (2 mL): 0.14 mL per spray, 4 sprays in each nostril, with repeat administration after 5 minutes of 4 sprays in each nostril.

Participants self-administer IN NAC up to 400 mg one to three times daily using the Aptar CPS Nasal Pump. On assessment days, IN NAC is administered by site staff. Participants are trained by site staff on the use of the Aptar CPS Nasal pump.

Safety and tolerability are monitored as outlined in TABLE 6. Participants experiencing treatment-limiting adverse effects are discontinued from the study. Participants return for a Follow-up Visit 7 days following the last dose of IP (Day 36±3 days) and receive a follow-up telephone call on Day 57 (±2 days) for safety assessment.

The effects of oral administration of NAC are also compared to IN NAC. 200 mg/mL of acetylcysteine as a 20% w/v solution is used. NAC is orally administered by diluting the NAC solution in a diet soft drink to a concentration of 5%. The oral dose studied is a 4,000 mg dose, which is prepared by adding 20 mL of a 20% NAC solution in 60 mL of diet soda to yield 80 mL of a 5% NAC solution.

TABLE 6

| | | | | | | | | |
|---|---|---|---|---|---|---|---|---|
| | Screening | | | Dosing | | | Study | |
| | visit¹ | Dosing | Dosing | Days 3-6 | Dosing | Post- | Follow-up | Telephone |
| | Day −28 | Day 1 | Day 2 | Days 3 | Day 7 | Dose | Visit | call |
| Activity | to −1 | Day 1 | Day 2 | to 6 | Day 7 | Day 8 | Day 14 | Day 35 |

Screening/Administrative/Other Assessments

| | | |
|---|---|---|
| Informed consent | X | |
| Demography | X | |
| Eligibility criteria | X | X |
| Medical and medica- | X | |
| tion history | | |
| Drug/alcohol | X | X |
| screen | | |
| Laboratory tests² | X | |
| Enrollment | | X |

Safety Assessments

| | | | | | | | | |
|---|---|---|---|---|---|---|---|---|
| Physical exam | X | | | | | | X | |
| Brief physical | | X | X | X | X | X | | |
| exam | | | | | | | | |
| Neurologic exam | X | | | | | | X | |
| Brief neurologic | | X | X | X | X | X | | |
| exam | | | | | | | | |
| Vital signs | X | | X | X | X | X | X | |
| Height | X | | | | | | | |
| Weight | X | | | | | | X | |
| 12-lead ECG³ | X | X | | | | | X | |
| Laboratory tests² | | X | | | | | X | |
| Urinalysis | X | | | | | | X | |
| VAS-tolerability | | X | X | X | X | | | |
| TNSS-M | X | X | X | X | X | X | X | X |
| Adverse event | X | X | X | X | X | X | X | X |
| monitoring | | | | | | | | |
| Concomitant | X | X | | | | X | X | X |
| meds | | | | | | | | |

IP Administration/MRS/Pharmacokinetic Assessments

| | | | | | |
|---|---|---|---|---|---|
| IP dosing⁴ | X | X | X | X | |
| MRS⁵ | X | X | | X | X |
| CSF NAC & | X | | | X | |
| GSH⁶ | | | | | |
| Plasma GSH, | X | X | | X | X |
| cysteine, free | | | | | |
| and total NAC⁷ | | | | | |
| RBC GSH/ | X | X | | X | X |
| GSSG⁷ | | | | | |

ABBREVIATIONS:
ECG, electrocardiogram;
GSH, glutathione GSH/GSSG, reduced-to-oxidized glutathione;
MRS, magnetic resonance spectroscopy;
NAC, N-acetylcysteine;
TNSS-M, Modified Total Nasal Symptom Scale;
VAS-T, Visual Analog Scale for Tolerability
¹Screening procedures must occur within 28 days of Day 1 IP dosing
²See Laboratory assessments for list of tests to be completed.
³ECG is performed pre-dose on Days 1 and 7
⁴BID or TID dosing depending on Parts 1&2.
⁵MRS pre-dose and 1, 3, 6 and 24 hours post-day 1 morning dose and post-day 7 morning dose
⁶CSF sampling on Days 1 and Day 7 following the 6-hour post-dose MRS
⁷Blood samples for NAC, cysteine, GSH and RBC GSH/GSSG drawn prior to each MRS session.

All medications, including over-the-counter medications, vitamins, and herbal supplements, taken during the 30 days prior to the first NAC administration are recorded and reviewed by the Investigator to determine whether the participant is suitable for inclusion in the study. Prior therapy or concomitant therapy with any medications, including both prescription and non-prescription drugs are discussed with the Investigator and Sponsor's MM before IP administration, except in the case of necessary treatment of AEs or where appropriate medical care necessitates that therapy begins before the Investigator can consult with the MM. The use of any NAC or investigational medical device within 30 days prior to Screening is prohibited. Paracetamol/acetaminophen (1-2 therapeutic doses per week) can be used for minor ailments during the course of the study, at the discretion of the Investigator, without prior consultation with Sponsor's MM.

### Example 7: Comparison of the Brain Bioavailability of in NAC with Different Participant Positions During Administration

FIG. 4 illustrates a schematic to study the effect of the subject's position during IP administration. ABBREVIATIONS: ECG, electrocardiogram; GSH/GSSG, reduced-to-oxidized glutathione; MRS, magnetic resonance spectroscopy; NAC, N-acetylcysteine; TNSS-M, modified total nasal symptom scale; VAS-T, Visual analog scale for Tolerability. [1] Screening procedures must occur within 28 days of study drug administration; [2] See laboratory assessments for list of tests to be completed; [3] Vital signs include temperature, pulse, and blood pressure, and are completed before and after each MRS session; [4] IN NAC to be given immediately following pre-dose scan, with dose (100, 200, or 400 mg) to be determined in single ascending dose study; [5] MRS to be completed pre-dose and 1, 3, 6, and 24 hours post-dose. The formulation, dose, and administration for IN NAC are determined based on the results of EXAMPLES 1-6. Participants are randomized in a 1:1 ratio to different sequences of IN NAC dosing parameters that differ in head positioning during IP administration. The participant receives IN NAC in either the supine position, with limited activity permitted following dosing, or in a seated position, after which the study participant is permitted to remain seated, stand or walk between MRS sessions. One cohort is assigned to supine administration of IP on Day 1 and seated administration on Day 8, with the other cohort assigned to the alternate sequence of dosing conditions.

Participants who receive IP in a supine position are required to remain recumbent, to the extent possible, from the time of the baseline scan to the completion of the 6-hour post-dose MRS. Participants who receive IP when seated may sit, stand or walk between MR sessions, and are encouraged to stand or walk for 10 minutes each hour from completion of the 1-hour post-dose MRS until completion of the 6-hour post-dose scan.

MRS is performed, and blood samples are collected to determine peripheral blood concentrations of GSH, cysteine, free and total NAC and RBG GSH/GSSG ratios prior to IP administration, MRS and blood sample analyses are repeated 1, 3, 6, and 24 hours post-dose. Changes from baseline in the relative levels of NAC-derived neurometabolites following IP administration are assessed with the participant in one of two positions during IN NAC administration: 1) Supine position—IP is administered with the participant supine, with participant to remain recumbent, to the extent possible, until after the 6-hour post-IP MRS; and 2) seated position—IP is administered with the participant seated, after which the participant is encouraged to stand or walk for at least 10 minutes per hour until completion of the 6-hour post-IP MRS. TABLE 7 shows the dose cohorts for comparison of participant positioning during NAC administration.

TABLE 7

| Cohort | Day 1 | Day 8 |
|---|---|---|
| 4A | Supine/limited ambulation | Seated/ambulation permitted |
| 4B | Seated/ambulation permitted | Supine/limited ambulation |

The NAC dose, formulation, and device for administration are determined based on the results of EXAMPLES 1-6. Safety and tolerability are monitored as outlined in TABLE 8. Participants experiencing treatment-limiting adverse effects are discontinued from the study. Participants return for a Follow-up Visit 7 days following the last dose of IP (Day 36±3 days) and receive a follow-up telephone call on Day 57 (±2 days) for safety assessment.

TABLE 8

| | | | | | |
|---|---|---|---|---|---|
| | Screening | | | Study | |
| | visit¹ | | | Follow-up | Telephone |
| | Day −28 | Dose 1 | Dose 2 | Visit | call |
| Activity | to −1 | Day 1 | Day 8 | Day 15 | Day 36 |

Screening/Administrative/Other Assessments

| | | | |
|---|---|---|---|
| Informed consent | X | | |
| Demography | X | | |
| Eligibility criteria | X | X | X |
| Medical/medica- | X | | |
| tion history | | | |
| Drug/alcohol | X | X | X |
| screen | | | |
| Laboratory tests² | X | | |
| Randomization | | X | |

Safety Assessments

| | | | | | |
|---|---|---|---|---|---|
| Physical exam | X | | | X | |
| Brief physical | | X | X | | |
| exam | | | | | |
| Neurologic exam | X | | | | X |
| Brief neurologic | | X | X | X | |
| exam | | | | | |
| Vital signs³ | X | X | X | X | |
| Height | X | | | | |
| Weight | X | | | X | |
| 12-lead ECG | X | | | X | |
| Laboratory tests² | | X | X | X | |
| Urinalysis | X | | | | |
| VAS-tolerability | | X | X | X | |
| TNSS-M | X | X | X | X | X |
| Adverse Event | X | X | X | X | X |
| Monitoring | | | | | |
| Concomitant | X | X | X | X | X |
| meds | | | | | |

Study Agent Administration/Pharmacokinetic Assessments

| | | |
|---|---|---|
| IP dosing | X | X |
| MRS⁴ | X | X |
| Plasma GSH, | X | X |
| cysteine, free | | |
| and total NAC⁵ | | |
| RBC GSH/ | X | X |
| GSSG⁵ | | |

¹Screening procedures must occur within 21 days of study drug administration.
²See Laboratory assessments for list of tests to be completed.
³Supine vital signs 5 minutes of rest include temperature, pulse, blood pressure on admission and before & after each MRS scan.
⁴MRS pre-dose and 1, 3, 6 and 24 hours post-dose
⁵Blood samples for NAC, cysteine, GSH and RBC GSH/GSSG drawn prior to each MR session.

### Example 8: Repeat Dosing Study

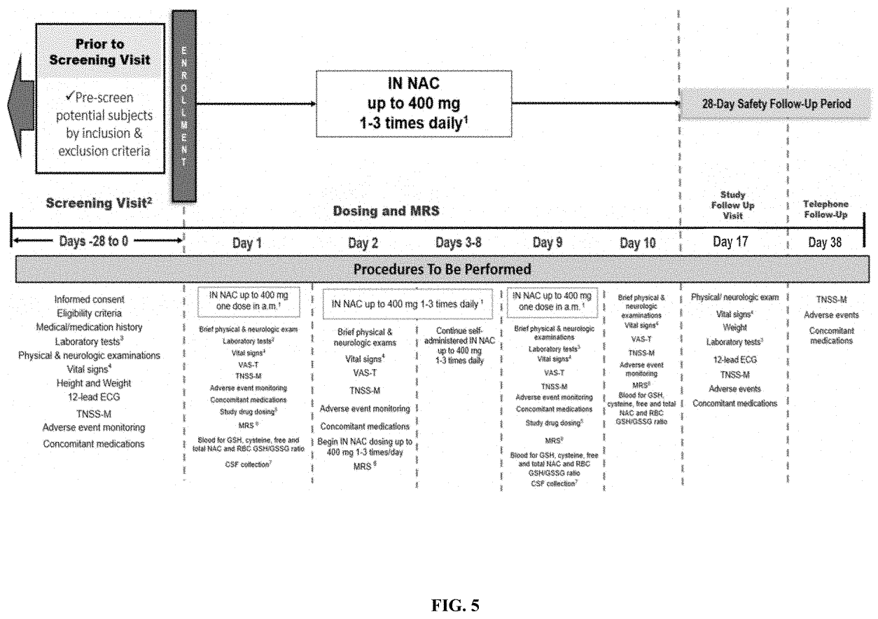

FIG. 5 illustrates a schematic to study the effect of repeat dosing of intranasal N-acetylcysteine administration. ABBREVIATIONS: CSF, cerebrospinal fluid; ECG, electrocardiogram; GSH/GSSG, reduced-to-oxidized glutathione; MRS, magnetic resonance spectroscopy; NAC, N-acetylcysteine; PK, pharmacokinetic; TNSS-M, modified total nasal symptom scale; VAS-T, Visual analog scale for Tolerability. [1] Parameters for IN NAC administration, including dose, device utilized, formulation, and other aspects of dosing will be determined based on results of the study; [2] Screening procedures must occur within 28 days of study drug administration; [3] See laboratory assessments for list of tests to be completed; [4] Vital signs include temperature, pulse, and blood pressure and are completed before and after each MRS session; [5] IP to be administered immediately following pre-dose scan; [6] MRS to be completed pre-dose and 1, 3, 6 hours on Day 1, with 24-hour post-IP MRS performed on the morning of Day 2 (prior to AM IP dose); [7] CSF to be collected 6 hours following morning dose on days 1 and 9; [8] MRS before and 1, 3, and 6 hours after IP dose on Day 9 with 24 hour post-IP MRS performed on the morning of Day 10._Ten healthy volunteers are assessed for the change from baseline in the relative levels of NAC-derived neurometabolites before and after 7 days of repeat dosing of IN NAC up to 400 mg one to three times daily. Ten study participants undergo MRS and other assessments before and after 7 days of repeat dosing of IN NAC up to 400 mg one to three times daily. The NAC dose, formulation, and administration are determined based on the results of EXAMPLES 1-6.

On Day 1, MRS is performed, and blood samples are collected for determination of peripheral blood concentrations of GSH, cysteine, free and total NAC, and RBG GSH/GSSG ratios prior to pre-dose and at 1, 3, and 6 hours following administration of IP. CSF samples are collected using a 22-gauge atraumatic needle to measure NAC concentration 6 hours after IN NAC dosing. On Day 2, a 24-hour post-dose MRS and other assessments are performed, and participants begin self-administered IN NAC at a dose of up to 400 mg one to three times daily. Participants continue self-administered IN NAC on days 3-8, for a total of seven days of repeat-dose. On Day 9, study participants return for IN NAC administration, MRS, and other assessments according to the same schedule as on Day 1. A 24-hour, post-dose MRS and collection of blood for laboratory tests are performed on Day 10.

Safety and tolerability are monitored as outlined in the Schedule of Assessments of TABLE 9. Participants experiencing treatment-limiting adverse effects are discontinued from the study. There is a Follow-up Visit 7 days following the last dose of IP (Day 17±3 days), and the subjects receive a follow-up telephone call on Day 38 (±2 days) for safety assessment.

TABLE 9

| | | | | | |
|---|---|---|---|---|---|
| | Screening | | | Study | |
| | visit¹ | | | Follow-up | Telephone |
| | Day −28 | Dose 1 | Dose 2 | Visit | call |
| Activity | to −1 | Day 1 | Day 8 | Day 15 | Day 36 |

Screening/Administrative/Other Assessments

| | | | |
|---|---|---|---|
| Informed consent | X | | |
| Demography | X | | |
| Eligibility criteria | X | X | X |
| Medical/medica- | X | | |
| tion history | | | |
| Drug/alcohol | X | X | X |
| screen | | | |
| Laboratory tests² | X | | |
| Randomization | | X | |

Safety Assessments

| | | | | | |
|---|---|---|---|---|---|
| Physical exam | X | | | X | |
| Brief physical | | X | X | | |
| exam | | | | | |
| Neurologic exam | X | | | | X |
| Brief neurologic | | X | X | X | |
| exam | | | | | |
| Vital signs³ | X | X | X | X | |
| Height | X | | | | |
| Weight | X | | | X | |
| 12-lead ECG | X | | | X | |
| Laboratory tests² | | X | X | X | |
| Urinalysis | X | | | | |
| VAS-tolerability | | X | X | X | |
| TNSS-M | X | X | X | X | X |
| Adverse Event | X | X | X | X | X |
| Monitoring | | | | | |
| Concomitant | X | X | X | X | X |
| meds | | | | | |

Study Agent Administration/Pharmacokinetic Assessments

| | | |
|---|---|---|
| IP dosing | X | X |
| MRS⁴ | X | X |
| Plasma GSH, | X | X |
| cysteine, free | | |
| and total NAC⁵ | | |
| RBC GSH/ | X | X |
| GSSG⁵ | | |

¹Screening procedures must occur within 21 days of study drug administration.
²See Laboratory assessments for list of tests to be completed.
³Supine vital signs 5 minutes of rest include temperature, pulse, blood pressure on admission and before & after each MRS scan.
⁴MRS pre-dose and 1, 3, 6 and 24 hours post-dose
⁵Blood samples for NAC, cysteine, GSH and RBC GSH/GSSG drawn prior to each MR session.

### Example 9: Comparison of IN, IV, and Oral Dosing of NAC

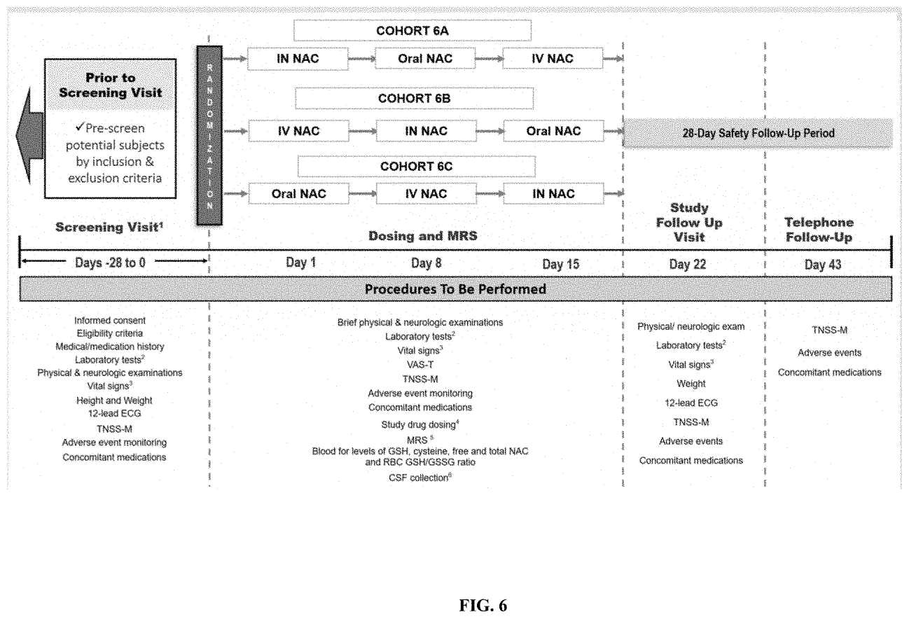

FIG. 6 illustrates a schematic to compare the effects of intranasal, intravenous, and oral administration of N-acetylcysteine. ABBREVIATIONS: ECG, electrocardiogram; GSH/GSSG, reduced-to-oxidized glutathione; MRS, magnetic resonance spectroscopy; NAC, N-acetylcysteine; PK, pharmacokinetic; TNSS-M, modified total nasal symptom scale; VAS-T, Visual analog scale for Tolerability. [1] Screening procedures must occur within 28 days of study drug administration; [2] See laboratory assessments for list of tests to be completed; [3] Vital signs include temperature, pulse, and blood pressure and are completed before and after each MRS session; [4] IN NAC to be given immediately following pre-dose scan, with dose (100, 200, or 400 mg) to be determined based on study results; [5] MRS to be completed pre-dose and 1, 3, 6, and 24 hours post-dose; [6] CSF to be collected 6 hours following IP dose on days 1, 8, and 15. The change from baseline in the relative levels of NAC-derived neurometabolites following administration of IN, oral, or IV NAC is determined. Twelve study participants are randomized in a 1:1:1 ratio to different sequences of weekly NAC dosing via 3 different formulations: IN NAC, a 4,000 mg oral NAC dose, or 150 mg/kg IV NAC. The 4,000 mg oral NAC dose is given as 20 mL of a 200 mg/mL acetylcysteine (20% w/v) solution. The IV NAC formulation is given as NAC at a dose of 150 mg/kg in 200 mL of sterile water, 0.45% normal saline, or 5% glucose in water. The sequence of administration in intervals is outlined in TABLE 10. The formulation, dose and administration for IN NAC are determined based on the results of EXAMPLES 1-6.

On each dosing day, MRS is performed, and blood samples are collected to determine peripheral blood concentrations of GSH, cysteine, free and total NAC, and RBG GSH/GSSG ratios prior to pre-dose and at 1, 3, 6, and 24 hours following IP administration. CSF samples are collected by lumbar puncture 6 hours after IP administration using a 22-gauge atraumatic needle. The CSF sample is used to measure NAC concentration 6 hours post-dose.

TABLE 10

| Cohort | Day 1 | Day 8 | Day 15 |
|---|---|---|---|
| 6A | Optimized IN | 4,000 mg oral | IV NAC 150 |
| | NAC dose | NAC dose | mg/kg |
| 6B | IV NAC 150 | Optimized IN | 4,000 mg oral |
| | mg/kg | NAC dose | NAC dose |
| 6C | 4,000 mg oral | IV NAC 150 | Optimized IN |
| | NAC dose | mg/kg | NAC dose |

Safety and tolerability are monitored by AE reporting, ECG, vital signs, physical and neurological examinations, and safety blood and urine tests. Tolerability of IN administration of IP is further assessed with a Visual Analog Scale for tolerability (VAS-T) and a Modified Total Nasal Symptom Score (TNSS-M). Participants experiencing treatment-limiting adverse effects are discontinued from the study. There is a Follow-up Visit 7 days following the last dose of IP (Day 22±3 days), and patients receive a follow-up telephone call on Day 43 (±2 days) for safety assessment. TABLE 11 shows the schedule of assessments for the comparison of IN, IV, and oral NAC administration.

TABLE 11

| | | | | | |
|---|---|---|---|---|---|
| | Screening | | | Study | |
| | visit¹ | | | Follow-up | Telephone |
| | Day −28 | Dose 1 | Dose 2 | Visit | call |
| Activity | to −1 | Day 1 | Day 8 | Day 15 | Day 36 |

Screening/Administrative/Other Assessments

| | | | |
|---|---|---|---|
| Informed consent | X | | |
| Demography | X | | |
| Eligibility criteria | X | X | X |
| Medical/medica- | X | | |
| tion history | | | |
| Drug/alcohol | X | X | X |
| screen | | | |
| Laboratory tests² | X | | |
| Randomization | | X | |

Safety Assessments

| | | | | | |
|---|---|---|---|---|---|
| Physical exam | X | | | X | |
| Brief physical | | X | X | | |
| exam | | | | | |
| Neurologic exam | X | | | | X |
| Brief neurologic | | X | X | X | |
| exam | | | | | |
| Vital signs³ | X | X | X | X | |
| Height | X | | | | |
| Weight | X | | | X | |
| 12-lead ECG | X | | | X | |
| Laboratory tests² | | X | X | X | |
| Urinalysis | X | | | | |
| VAS-tolerability | | X | X | X | |
| TNSS-M | X | X | X | X | X |
| Adverse Event | X | X | X | X | X |
| Monitoring | | | | | |
| Concomitant | X | X | X | X | X |
| meds | | | | | |

Study Agent Administration/Pharmacokinetic Assessments

| | | |
|---|---|---|
| IP dosing | X | X |
| MRS⁴ | X | X |
| Plasma GSH, | X | X |
| cysteine, free | | |
| and total NAC⁵ | | |
| RBC GSH/ | X | X |
| GSSG⁵ | | |

¹Screening procedures must occur within 21 days of study drug administration.
²See Laboratory assessments for list of tests to be completed.
³Supine vital signs 5 minutes of rest include temperature, pulse, blood pressure on admission and before & after each MRS scan.
⁴MRS pre-dose and 1, 3, 6 and 24 hours post-dose
⁵Blood samples for NAC, cysteine, GSH and RBC GSH/GSSG drawn prior to each MR session.

### Example 10: Optimization of Therapeutic Agent and Drug Delivery Using MEGA-PRESS

A patient with Parkinson's disease is treated with intranasal NAC, NACA, NAC derivative, NAC metabolite, NAC congener, or D-NAC, or a pharmaceutically-acceptable salt thereof. MEGA-PRESS is used to quantify the amount of GSH in the substantia nigra and stratum regions of the brain. The therapeutic agent, dose, dosing interval, and dose delivery system are optimized to deliver the greatest amount of GSH to the substantia nigra and stratum regions of the brain to treat Parkinson's disease.

A patient with hemorrhagic stroke is treated with intranasal NAC, NACA, NAC derivative, NAC metabolite, NAC congener, or D-NAC, or a pharmaceutically-acceptable salt thereof. MEGA-PRESS is used to quantify the amount of NAC in regions of the brain. The therapeutic agent, dose, dosing interval, and dose delivery system are optimized to deliver the greatest concentration of NAC at the site of hemorrhage.

### Example 11: Regional and Temporal Changes in NAC Metabolites

One mL of a 200 mg/mL NAC solution was delivered, 0.5 mL per nostril, intranasally to 5 subjects using a Teleflex mucosal atomizer (MAD). MRS analysis was used to determine the concentration of GSH, NAA, water, and creatine pre-dose, 1 hour, 3 hours, and 6 hours after administration of NAC. Ratios of GSH/water (I.U.), GSH/creatine, NAA/water, and NAA/creatine were determined.

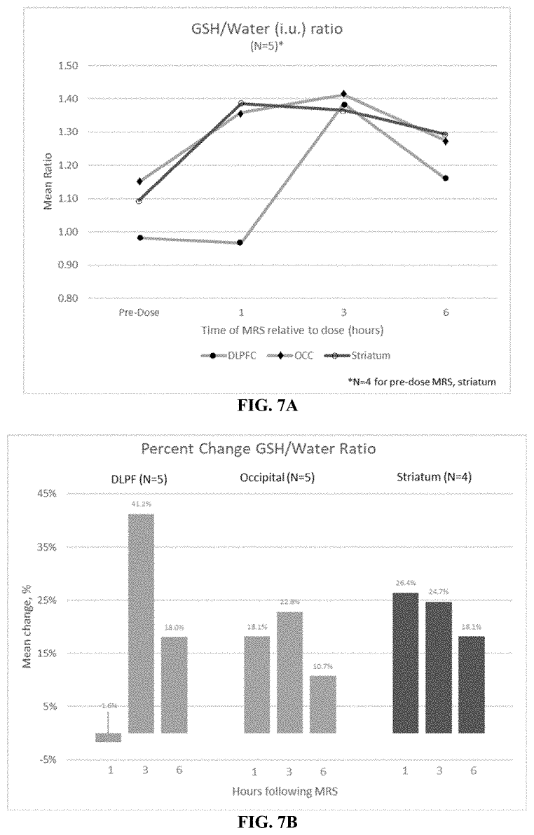

FIG. 7A shows the change in GSH/water (I.U.) ratio in the dorsolateral prefrontal cortex (DLPF), occipital lobe (OCC), and striatum regions of the brain. FIG. 7B shows the percent change in GSH/water ratio in the DLPF, OCC, and striatum.

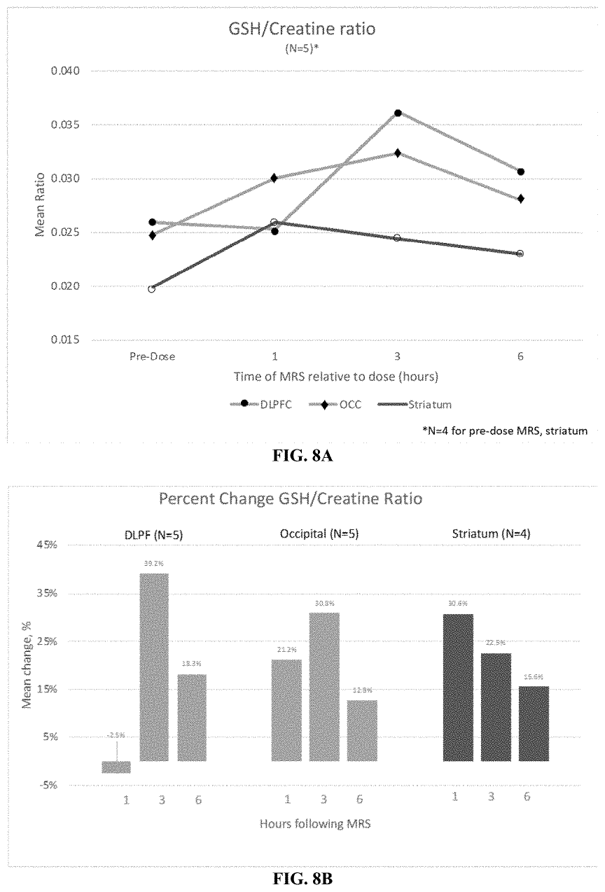

FIG. 8A shows the change in GSH/creatine ratio in the dorsolateral prefrontal cortex (DLPF), occipital lobe (OCC), and striatum regions of the brain. FIG. 8B shows the percent change in GSH/creatine ratio in the DLPF, OCC, and stratum regions of the brain.

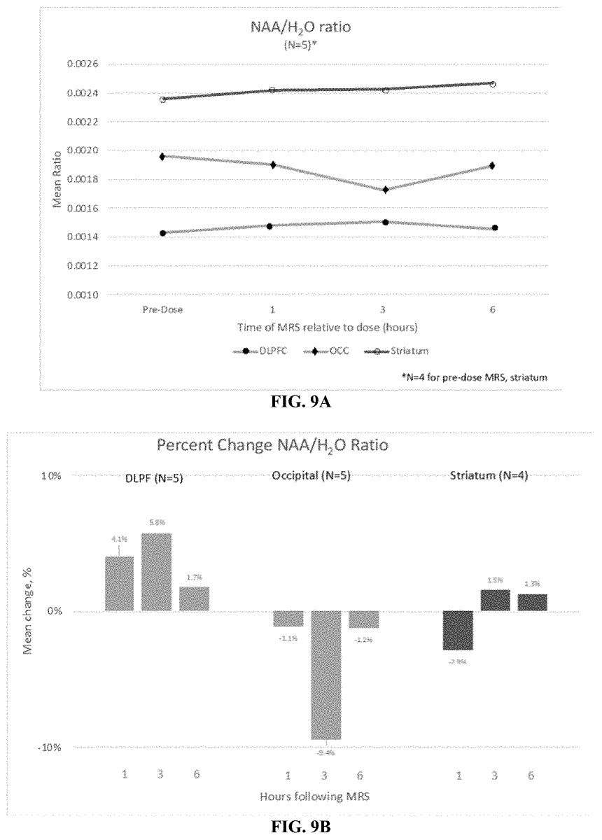

FIG. 9A shows the N-acetyl aspartate (NAA)/water ratio in the dorsolateral prefrontal cortex (DLPF), occipital lobe (OCC), and striatum regions of the brain. FIG. 9B shows the percent change in the NAA/water ratio in the dorsolateral prefrontal cortex (DLPF), occipital lobe (OCC), and striatum regions of the brain.

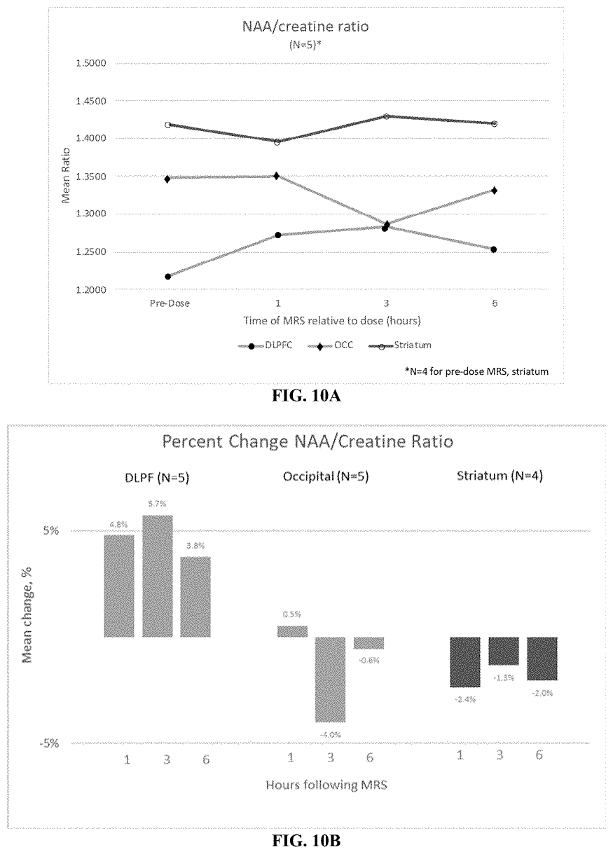

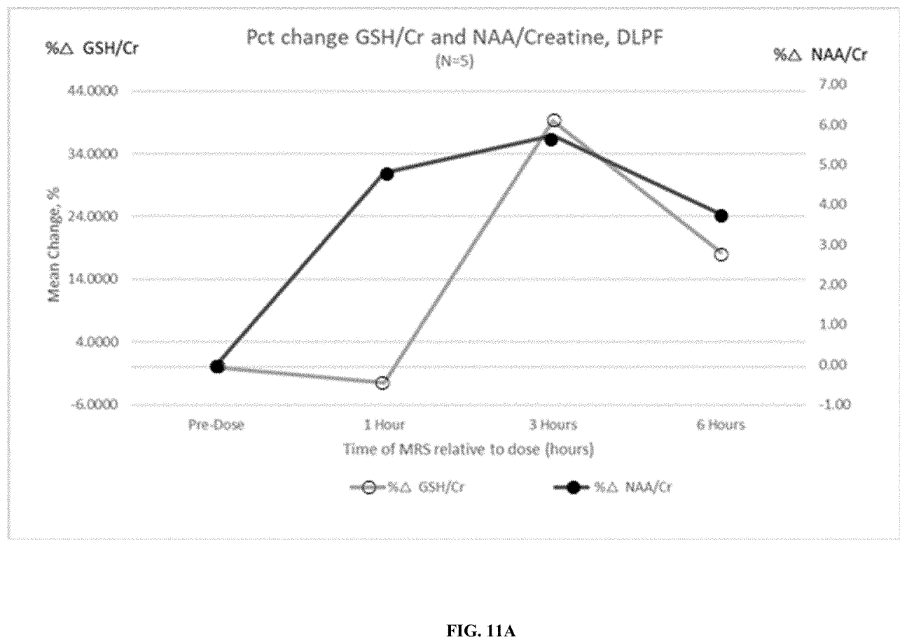

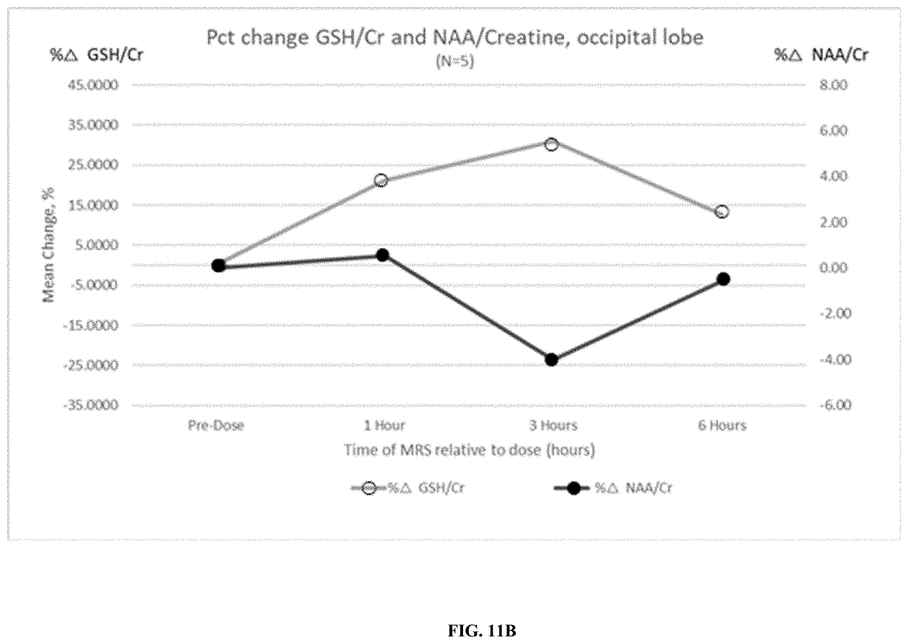

FIG. 10A shows the N-acetyl aspartate (NAA)/creatine ratio in the dorsolateral prefrontal cortex (DLPF), occipital lobe (OCC), and striatum regions of the brain. FIG. 10B shows the percent change in the NAA/creatine ratio in the dorsolateral prefrontal cortex (DLPF), occipital lobe (OCC), and striatum regions of the brain.

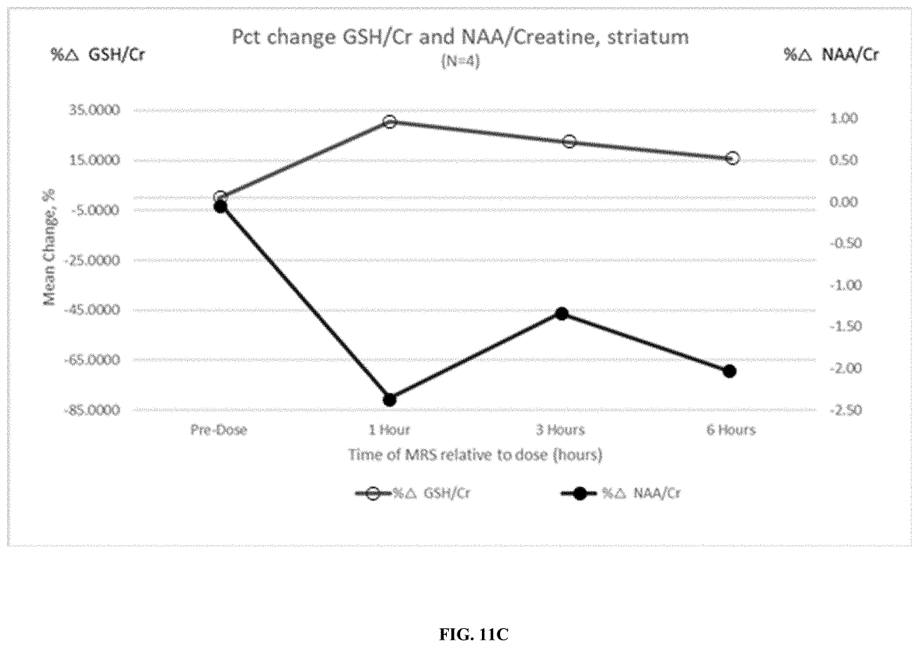

FIG. 11A shows the percent change of GSH/creatine and percent change of NAA/creatine in the dorsolateral prefrontal cortex (DLPF) region of the brain. FIG. 11B shows the percent change of GSH/creatine and percent change of NAA/creatine in the occipital lobe (OCC) region of the brain. FIG. 11C shows the percent change of GSH/creatine and percent change of NAA/creatine in the striatum region of the brain.

### Embodiments

The following non-limiting embodiments provide illustrative examples of the invention, but do not limit the scope of the invention.

Embodiment 1. A method of treating a condition comprising: a) administering to a subject in need thereof a therapeutically-effective amount of a therapeutic agent, wherein the administering is intranasal; and b) after the administering, quantifying a concentration of glutathione in a brain region by magnetic resonance spectroscopy.

Embodiment 2. The method of embodiment 1, wherein the therapeutic agent is N-acetylcysteine (NAC).

Embodiment 3. The method of embodiment 1, wherein the therapeutic agent is NAC amide (NACA).

Embodiment 4. The method of embodiment 1, wherein the therapeutic agent is a NAC derivative or a pharmaceutically-acceptable salt thereof.

Embodiment 5. The method of embodiment 1, wherein the therapeutic agent is a NAC congener or a pharmaceutically-acceptable salt thereof.

Embodiment 6. The method of embodiment 1, wherein the therapeutic agent is a NAC dendrimer or a pharmaceutically-acceptable salt thereof.

Embodiment 7. The method of any one of embodiments 1-6, wherein the condition is a brain condition.

Embodiment 8. The method of any one of embodiments 1-7, wherein the brain condition is mild traumatic brain injury (mTBI).

Embodiment 9. The method of any one of embodiments 1-7, wherein the brain condition is a cancer.

Embodiment 10. The method of any one of embodiments 1-7, wherein the brain condition is a central nervous system (CNS) disorder.

Embodiment 11. The method of any one of embodiments 1-7 and 10, wherein the CNS disorder is Parkinson's disease.

Embodiment 12. The method of any one of embodiments 1-11, wherein the therapeutically-effective amount is from about 100 mg to about 400 mg.

Embodiment 13. The method of any one of embodiments 1-12, wherein the therapeutically-effective amount is about 100 mg.

Embodiment 14. The method of any one of embodiments 1-12, wherein the therapeutically-effective amount is about 200 mg.

Embodiment 15. The method of any one of embodiments 1-12, wherein the therapeutically-effective amount is about 400 mg.

Embodiment 16. The method of any one of embodiments 1-15, wherein the therapeutic agent is administered using a nasal pump.

Embodiment 17. The method of any one of embodiments 1-15, wherein the therapeutic agent is administered using an atomizer.

Embodiment 18. The method of any one of embodiments 1-17, wherein the administering is performed in a supine position.

Embodiment 19. The method of any one of embodiments 1-17, wherein the administering is performed in a seated position.

Embodiment 20. The method of any one of embodiments 1-19, further comprising quantifying an amount of free NAC in a plasma sample.

Embodiment 21. The method of any one of embodiments 1-20, further comprising quantifying an amount of total NAC in a plasma sample.

Embodiment 22. The method of any one of embodiments 1-21, further comprising quantifying an amount of plasma GSH.

Embodiment 23. The method of any one of embodiments 1-22, further comprising quantifying a ratio of reduced GSH to oxidized GSH (GSH/GSSG).

Embodiment 24. The method of any one of embodiments 1-23, further comprising quantifying an amount of NAC or a NAC metabolite in a cerebrospinal fluid sample.

Embodiment 25. The method of any one of embodiments 1-24, wherein the administering is once daily.

Embodiment 26. The method of any one of embodiments 1-24, wherein the administering is twice daily.

Embodiment 27. The method of any one of embodiments 1-24, wherein the administering is three time daily.

Embodiment 28. The method of any one of embodiments 1-27, wherein the administering is repeated at least one time.

Embodiment 29. The method of any one of embodiments 1-28, wherein the administering is repeated once.

Embodiment 30. The method of any one of embodiments 1-28, wherein the administering is repeated twice.

Embodiment 31. The method of any one of embodiments 1-30, wherein the administering is repeated after about 7 days.

Embodiment 32. The method of any one of embodiments 1-31, wherein the therapeutic agent is in a pharmaceutical composition.

Embodiment 33. The method of any one of embodiments 1-32, wherein the therapeutic agent is an aqueous solution.

Embodiment 34. The method of any one of embodiments 1-33, wherein the pharmaceutical composition comprises from about 5% to about 40% of the therapeutic agent.

Embodiment 35. The method of any one of embodiments 1-34, wherein the pharmaceutical composition comprises about 20% of the therapeutic agent.

Embodiment 36. The method of any one of embodiments 1-35, wherein the pharmaceutical composition further comprises a pharmaceutically-acceptable excipient.

Embodiment 37. The method of any one of embodiments 1-36, wherein the pharmaceutically-acceptable excipient is disodium edate.

Embodiment 38. The method of any one of embodiments 1-37, wherein the pharmaceutically-acceptable excipient is sodium hydroxide.

Embodiment 39. The method of any one of embodiments 1-38, wherein the pharmaceutical composition further comprises a pH adjustor.

Embodiment 40. The method of embodiment 39, wherein the pH adjustor is hydrochloric acid.

Embodiment 41. The method of any one of embodiments 1-40, wherein the brain region is a cerebrum.

Embodiment 42. The method of any one of embodiments 1-40, wherein the brain region is a brainstem.

Embodiment 43. The method of any one of embodiments 1-40, wherein the brain region is a cerebellum.

Embodiment 44. The method of any one of embodiments 1-40, wherein the brain region is a pons.

Embodiment 45. The method of any one of embodiments 1-40, wherein the brain region is a medulla.

Embodiment 46. The method of any one of embodiments 1-40, wherein the brain region is a frontal lobe.

Embodiment 47. The method of any one of embodiments 1-40, wherein the brain region is a parietal lobe.

Embodiment 48. The method of any one of embodiments 1-40, wherein the brain region is a occipital lobe.

Embodiment 49. The method of any one of embodiments 1-40, wherein the brain region is a temporal lobe.

Embodiment 50. The method of any one of embodiments 1-40, wherein the brain region is the left dorsal striatum.

Embodiment 51. The method of any one of embodiments 1-40, wherein the brain region is a occipital cortex.

Embodiment 52. The method of any one of embodiments 1-40, wherein the brain region is a dorsolateral prefrontal cortex (DLPF).

Embodiment 53. The method of any one of embodiments 1-52, further comprising determining a change in the concentration of glutathione in the brain region over a period of time.

Embodiment 54. The method of any one of embodiments 1-53, wherein the administering increases the concentration of glutathione by from about 20% to about 300%.

Embodiment 55. The method of any one of embodiments 1-54, wherein the administering increases the concentration of glutathione by about 30%.

Embodiment 56. The method of any one of embodiments 1-54, wherein the administering increases the concentration of glutathione by about 50%.

Embodiment 57. The method of any one of embodiments 1-54, wherein the administering increases the concentration of glutathione by about 100%.

Embodiment 58. A method of treating a condition comprising: a) administering to a subject in need thereof a therapeutically-effective amount of a therapeutic agent; and b) after the administering, quantifying a concentration of the therapeutic agent or a metabolite of the therapeutic agent in a brain region of the subject by at least two magnetic resonance spectroscopy signals.

Embodiment 59. The method of embodiment 58, wherein the therapeutic agent is N-acetylecysteine (NAC).

Embodiment 60. The method of embodiment 58, wherein the therapeutic agent is NAC amide (NACA).

Embodiment 61. The method of embodiment 58, wherein the therapeutic agent is a NAC derivative.

Embodiment 62. The method of embodiment 58, wherein the therapeutic agent is a NAC congener or a pharmaceutically-acceptable salt thereof.

Embodiment 63. The method of embodiment 58, wherein the therapeutic agent is a NAC dendrimer or a pharmaceutically-acceptable salt thereof.

Embodiment 64. The method of any one of embodiments 58-63, wherein the metabolite of the therapeutic agent is glutathione.

Embodiment 65. The method of any one of embodiments 58-64, wherein the condition is a brain condition.

Embodiment 66. The method of embodiment 58-64, wherein the condition is mild traumatic brain injury (mTBI).

Embodiment 67. The method of embodiment 58-64, wherein the condition is cancer.

Embodiment 68. The method of embodiment 58-64, wherein the condition is hemorrhagic stroke.

Embodiment 69. The method of embodiment 58-64, wherein the condition is a central nervous system (CNS) disorder.

Embodiment 70. The method of embodiment 69, wherein the CNS disorder is Parkinson's disease.

Embodiment 71. The method of any one of embodiments 58-70, wherein the therapeutically-effective amount is from about 100 mg to about 400 mg.

Embodiment 72. The method of any one of embodiments 58-71, wherein the therapeutically-effective amount is about 100 mg.

Embodiment 73. The method of any one of embodiments 58-71, wherein the therapeutically-effective amount is about 200 mg.

Embodiment 74. The method of any one of embodiments 58-71, wherein the therapeutically-effective amount is about 400 mg.

Embodiment 75. The method of any one of embodiments 58-74, wherein the administering is by a nasal pump.

Embodiment 76. The method of any one of embodiments 58-74, wherein the administering is by an atomizer.

Embodiment 77. The method of any one of embodiments 58-77, wherein the administering is performed with the subject in a supine position.

Embodiment 78. The method of any one of embodiments 58-77, wherein the administering is performed with the subject in a seated position.

Embodiment 79. The method of any one of embodiments 58-78, further comprising obtaining a plasma sample of the subject after the administering and quantifying an amount of free NAC in a plasma sample.

Embodiment 80. The method of any one of embodiments 58-79, further comprising obtaining a plasma sample of the subject after the administering and quantifying an amount of total NAC in a plasma sample.

Embodiment 81. The method of any one of embodiments 58-80, further comprising obtaining a plasma sample of the subject after the administering and quantifying an amount of GSH in the plasma sample.

Embodiment 82. The method of any one of embodiments 58-81, further comprising quantifying a ratio of reduced GSH to oxidized GSH (GSH/GSSG) in the brain region after the administering.

Embodiment 83. The method of any one of embodiments 58-82, further comprising obtaining a cerebrospinal fluid sample of the subject after the administering and quantifying an amount of NAC or a NAC metabolite in the cerebrospinal fluid sample.

Embodiment 84. The method of any one of embodiments 58-83, wherein the administering is once daily.

Embodiment 85. The method of any one of embodiments 58-84, wherein the administering is twice daily.

Embodiment 86. The method of any one of embodiments 58-85, wherein the administering is three time daily.

Embodiment 87. The method of any one of embodiments 58-86, wherein the administering is repeated at least one time.

Embodiment 88. The method of any one of embodiments 58-87, wherein the administering is repeated once.

Embodiment 89. The method of any one of embodiments 58-87, wherein the administering is repeated twice.

Embodiment 90. The method of any one of embodiments 58-89, wherein the administering is repeated after about 7 days.

Embodiment 91. The method of any one of embodiments 58-90, wherein the therapeutic agent is in a pharmaceutical composition.

Embodiment 92. The method of any one of embodiments 58-91, wherein the therapeutic agent is an aqueous solution.

Embodiment 93. The method of any one of embodiments 58-92, wherein from about 5% to about 40% of the pharmaceutical composition is the therapeutic agent.

Embodiment 94. The method of any one of embodiments 58-93, wherein about 20% of the pharmaceutical composition is the therapeutic agent.

Embodiment 95. The method of any one of embodiments 58-94, wherein the pharmaceutical composition further comprises a pharmaceutically-acceptable excipient.

Embodiment 96. The method of embodiment 95, wherein the pharmaceutically-acceptable excipient is disodium edate.

Embodiment 97. The method of embodiment 96, wherein the pharmaceutically-acceptable excipient is sodium hydroxide.

Embodiment 98. The method of any one of embodiments 58-97, wherein the pharmaceutical composition further comprises a pH adjustor.

Embodiment 99. The method of embodiment 98, wherein the pH adjustor is hydrochloric acid.

Embodiment 100. The method of any one of embodiments 58-99, wherein the brain region is a cerebrum.

Embodiment 101. The method of any one of embodiments 58-99, wherein the brain region is a brainstem.

Embodiment 102. The method of any one of embodiments 58-99, wherein the brain region is a cerebellum.

Embodiment 103. The method of any one of embodiments 58-99, wherein the brain region is a pons.

Embodiment 104. The method of any one of embodiments 58-99, wherein the brain region is a medulla.

Embodiment 105. The method of any one of embodiments 58-99, wherein the brain region is a frontal lobe.

Embodiment 106. The method of any one of embodiments 58-99, wherein the brain region is a parietal lobe.

Embodiment 107. The method of any one of embodiments 58-99, wherein the brain region is a occipital lobe.

Embodiment 108. The method of any one of embodiments 58-99, wherein the brain region is a temporal lobe.

Embodiment 109. The method of any one of embodiments 58-99, wherein the brain region is a left dorsal striatum.

Embodiment 110. The method of any one of embodiments 58-99, wherein the brain region is an occipital cortex.

Embodiment 111. The method of any one of embodiments 58-99, wherein the brain region is a dorsolateral prefrontal cortex (DLPF).

Embodiment 112. The method of any one of embodiments 58-99, wherein the brain region is a substantia nigra.

Embodiment 113. The method of any one of embodiments 58-99, wherein the brain region is a striatum.

Embodiment 114. The method of any one of embodiments 58-113, further comprising determining a change in a concentration of glutathione in the brain region of the subject after administration over a period of time.

Embodiment 115. The method of any one of embodiments 58-114, further comprising determining a change in a concentration of NAC in the brain region of the subject after administration over a period of time.

Embodiment 116. The method of any one of embodiments 58-115, wherein the administering increases a concentration of glutathione in the brain region by from about 20% to about 300%.

Embodiment 117. The method of any one of embodiments 58-116, wherein the administering increases a concentration of NAC in the brain region by from about 20% to about 300%.

Embodiment 118. The method of any one of embodiments 58-117, wherein the administering increases a concentration of glutathione in the brain region by about 30%.

Embodiment 119. The method of any one of embodiments 58-117, wherein the administering increases a concentration of NAC in the brain region by about 30%.

Embodiment 120. The method of any one of embodiments 58-117, wherein the administering increases a concentration of glutathione in the brain region by about 50%.

Embodiment 121. The method of any one of embodiments 58-117, wherein the administering increases a concentration of NAC in the brain region by about 50%.

Embodiment 122. The method of any one of embodiments 58-117, wherein the administering increases a concentration of glutathione in the brain region by about 100%.

Embodiment 123. The method of any one of embodiments 58-117, wherein the administering increases a concentration of NAC in the brain region by about 100%.

What is claimed is:

1. A method of treating a condition comprising:
a) administering to a subject in need thereof a therapeutically-effective amount of a therapeutic agent, wherein the administering is intranasal; and
b) after the administering, quantifying a concentration of NAC or glutathione in a brain region by magnetic resonance spectroscopy.

2. The method of claim 1, wherein the therapeutic agent is N-acetylcysteine (NAC) or a pharmaceutically-acceptable salt thereof.

3. The method of claim 1, wherein the therapeutic agent is a NAC derivative.

4. The method of claim 1, wherein the condition is a brain condition.

5. The method of claim 4, wherein the brain condition is mild traumatic brain injury.

6. The method of claim 4, wherein the brain condition is a cancer.

7. The method of claim 4, wherein the brain condition is a central nervous system (CNS) disorder.

8. The method of claim 6, wherein the CNS disorder is Parkinson's disease.

9. The method of claim 1, wherein the therapeutically-effective amount is from about 100 mg to about 400 mg.

10. The method of claim 1, wherein the therapeutic agent is administered using a nasal pump.

11. The method of claim 1, wherein the therapeutic agent is administered using an atomizer.

12. The method of claim 1, wherein the administering is repeated at least one time.

13. The method of claim 1, wherein the therapeutic agent is in a pharmaceutical composition, wherein the pharmaceutical composition further comprises a pharmaceutically-acceptable excipient.

14. The method of claim 12, wherein the therapeutic agent is an aqueous solution.

15. The method of claim 1, wherein the brain region is a cerebrum.

16. The method of claim 1, wherein the brain region is a frontal lobe.

17. The method of claim 1, wherein the brain region is a occipital lobe.

18. The method of claim 1, wherein the brain region is the occipital cortex.

19. The method of claim 1, further comprising determining a change in the concentration of glutathione in the brain region over a period of time.

20. The method of claim 19, wherein the administering increases the concentration of glutathione by from about 20% to about 300%.
